(19) United States
(12) Patent Application Publication
(10) Pub. No.: US 2009/0087590 A1
(43) Pub. Date: Apr. 2, 2009

(54) OPTICALLY ANISOTROPIC FILM, METHOD OF PRODUCING THE SAME, AND LIQUID CRYSTAL DISPLAY DEVICE USING THE SAME

(75) Inventors: Yasuhiro Aiki, Minami-ashigara-shi (JP); Shinichi Morishima, Minami-ashigara-shi (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Minato-ku (JP)

(21) Appl. No.: 12/237,520

(22) Filed: Sep. 25, 2008

(30) Foreign Application Priority Data
Sep. 28, 2007 (JP) 2007-255232

(51) Int. Cl. C09K 19/06; C07C 69/76; C08G 63/16; B05D 5/12; B05D 3/06; C08G 63/688; C09K 11/06

(52) U.S. Cl. 428/1.1; 560/59; 528/302; 528/293; 252/301.16; 427/595; 427/532

(57) ABSTRACT

Disclosed is an optically anisotropic film comprising at least one compound having a partial structure represented by formula (1): where, each of R¹, R² and R³ independently represent a substituent; X represents a divalent linking group; “A” represents —COO—, —OCO—, or a substituted or non-substituted phenylene group, oxadiazole group or alkynylene group; Z represents a substituted or non-substituted alkyl group or aryl group; each of n1, n2 and n3 represents an integer of 0 to 4; and each of l, m and n represents an integer of 0 to 4.

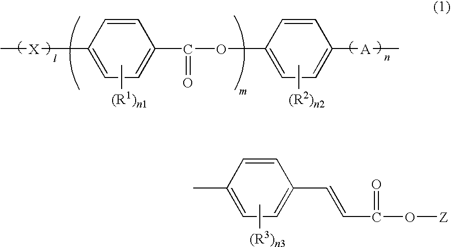

### CROSS-REFERENCE TO RELATED APPLICATIONS

This application claims benefit of priority under 35 U.S.C. 119 to Japanese Patent Application No. 2007-255232 filed on Sep. 28, 2007; and the entire contents of the application are incorporated herein by reference.

### BACKGROUND OF THE INVENTION

1. Field of the Invention

The present invention relates to an optically anisotropic film, a method of producing the same, and a liquid crystal display device using the same.

2. Related Art

There have been proposed various types of liquid crystal display devices. Among those, a VA (vertically aligned) mode display device has been proven to be a wide viewing angle mode display capable of omni-directionally achieving desirable contrast viewing-angle characteristics, and has already been as household television sets. In recent years, large-size displays of 30 inches or larger have been launched. In the VA-mode liquid crystal display devices, optically anisotropic film or the like, having various characteristics, have been used for optical compensation, for the purpose of reducing leakage of light and color shift observed in oblique directions the black state.

For example, as an optical compensation sheet contributive to improvement in color-viewing angle characteristics of the VA-mode liquid crystal display devices, a retardation plate formed of polycarbonate having predetermined optical characteristics has been proposed (Japanese Laid-Open Patent Publication No. 2004-37837).

Other proposals have been made on systems providing independent compensation for each of three colors of R, G and B (GB2394718, Japanese Laid-Open Patent Publication Nos. 2004-240102, 2005-4124, 2005-24919, 2005-24920, 2006-78647, and 2006-64858). Such systems may typically be realized by patterning a retardation layer together with a color filter and so forth in a liquid crystal cell. However, patterning of the retardation layer needs complicated steps, and, a patterned retardation layer may be prepared, for example, according to a method comprising formation of an alignment layer, rubbing the alignment layer, applying a polymerizable liquid crystal composition to a rubbed surface, alignment, fixing, formation of a resist layer for patterning the retardation layer, etching, and removal of the resist layer. It is difficult to prepare a retardation layer having optically uniform retardation characteristics according to such a complicated method. And in such a method, a retardation layer may be subjected to the heat and the solvent in the patterning treatment employing photoresist, and therefore retardation of the layer may be occasionally changed before and after the etching.

On the other hand, as a material for the retardation film, there has been proposed a birefringence-inducing material. One example of such a material is a material containing naphthyl acryloyl or its derivatives or biphenyl acryloyl or its derivatives; and birefringence is induced in the material due to molecular motion and subsequent molecular orientation generated by irradiating the material with light or heat (JPA Nos. 2004-258426 and 2006-308878).

### SUMMARY OF THE INVENTION

By using the birefringence-inducing material, the retardation layer having predetermined optical characteristics may be formed in micro-regions corresponded to the individual pixels in the liquid crystal cell, without using any patterning technique. However, examinations by the present inventors revealed that the materials proposed in JPA Nos. 2004-258426 and 2006-308878 sometimes failed in obtaining desired retardation necessary for the optical compensation. It was also found that retardation of the layer was changed, due to the treatments such as heating, solvent treatment and so forth involved in the process of producing a liquid crystal cell.

It is therefore an object of the present invention to provide a novel optically anisotropic film, a method of producing the same, and a polymer compound used for the production, which are useful for optical compensation and so forth of liquid crystal display devices.

It is another object of the present invention to provide an optically anisotropic film readily formable in a liquid crystal cell, and suppressed in fluctuation of the optical characteristics.

It is still another object of the present invention to provide a liquid crystal display device in which the liquid crystal cell is optically compensated in an exact manner, excellent in the productivity, and improved in the color-viewing angle characteristics.

The means for achieving the abovementioned objects are as follows.

[1] An optically anisotropic film comprising at least one compound having a partial structure represented by formula (1) below:

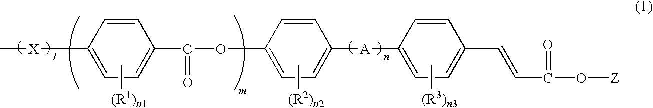

where, each of R¹, R² and R³ independently represents a substituent; X represents a divalent linking group selected from Linking Group I shown below, or a divalent linking group formed by combining two or more species selected from Linking Group I shown below; “A” represents —COO—, —OCO—, or a substituted or non-substituted phenylene group, oxadiazole group or alkynylene group; Z represents a substituted or non-substituted alkyl group or aryl group; each of n1, n2 and n3 represents an integer of 0 to 4; and each of l, m and n represents an integer of 0 to 4;

### Linking Group I:

single bond, —O—, —CO—, —NR⁶— (R⁶ represents a hydrogen atom, alkyl group or aryl group), —S—, —SO₂—, —P(═O)(OR⁷)— (R⁷ represents an alkyl group or aryl group), alkylene group and arylene group.

[2] The optically anisotropic film as set forth in [1], wherein said compound is a polymer compound having the partial structure represented by formula (1) in the side chain(s) thereof.
[3] The optically anisotropic film as set forth in [2], wherein said polymer compound comprises a repeating unit represented by formula (2):

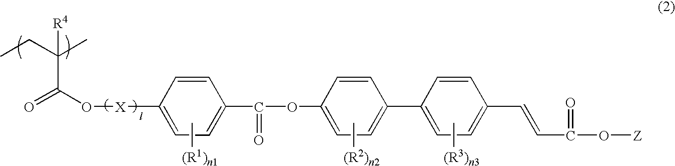

where, R⁴ represents a hydrogen atom or substituent, and other symbols are used for the same meaning with those in formula (1).

[4] The optically anisotropic film as set forth in [2] or [3], wherein said polymer compound further comprises a repeating unit represented by formula (5) and/or formula (7) below:

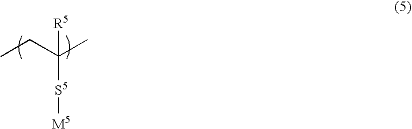

where, R⁵ represents a hydrogen atom or substituent, S⁵ represents a divalent linking group, and M⁵ represents a mesogen group;

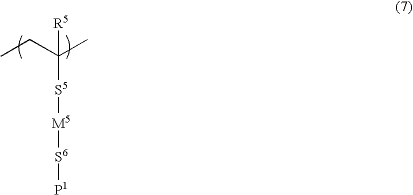

where, R⁵ represents a hydrogen atom or substituent, S⁵ represents a divalent linking group, M⁵ represents a mesogen group, S⁶ represents a divalent linking group, and P¹ represents a polymerizable group.

[5] The optically anisotropic film as set forth in any one of [1] to [4], formed of a composition comprising at least said compound irradiated with polarized light.
[6] The optically anisotropic film as set forth in [5], having Re(550), which is retardation in plane at 550 nm, is 20 nm to 300 nm.
[7] The optically anisotropic film as set forth in [5] or [6], being a positive A-plate.
[8] The optically anisotropic film as set forth in [5] or [6], having an Nz value, where Nz=Rth(550)/Re(550)+0.5, Rth(550) is retardation along thickness direction at 550 nm, and Re(550) is retardation in plane at 550 nm, of 1.1 to 7.0.
[9] The optically anisotropic film as set forth in any one of [1] to [4], formed of a composition comprising at least said compound irradiated with polarized light on a rubbed surface.
[10] The optically anisotropic film as set forth in [9], having an Nz value, where Nz=Rth(550)/Re(550)+0.5, Rth(550) is retardation along thickness direction at 550 nm, and Re(550) is retardation in plane at 550 nm, of 1.1 to 7.0.
[11] The optically anisotropic film as set forth in [5] or [6], having an Nz value, where Nz=Rth(550)/Re(550)+0.5, Rth(550) is retardation along thickness direction at 550 nm, and Re(550) is retardation in plane at 550 nm, of 0.1 to 0.9.
[12] A liquid crystal cell substrate, comprising a substrate and an optically anisotropic film as set forth in any one of [1] to [11].
[13] A liquid crystal display device comprising an optically anisotropic film as set forth in any one of [1] to [11].
[14] The liquid crystal display device as set forth in [13], being a VA-mode liquid crystal display device.
[15] The liquid crystal display device as set forth in [13], being an IPS-mode liquid crystal display device.
[16] The liquid crystal display device as set forth in [13] or
[14], wherein the optically anisotropic film is disposed in a liquid crystal cell.
[17] The liquid crystal display device as set forth in [16], where the optically anisotropic film is disposed in a liquid crystal cell, as being formed in the regions corresponded to the individual pixels.
[18] The liquid crystal display device as set forth in any one of [13] to [17], comprising an optically anisotropic film as set forth in [7] as a first optically anisotropic layer, and a second optically anisotropic layer having Rth (550) of 20 to 300 nm.
[19] A method of producing an optically anisotropic film comprising irradiating a composition with polarized light, so that birefringence develops in the composition,

wherein the composition comprises at least one compound having a partial structure represented by formula (1) defined in [1].

[20] A method of producing an optically anisotropic film comprising disposing a composition on a rubbed surface; and irradiating the composition with polarized light in a direction different from the rubbing direction of said rubbed surface, so that birefringence develops in the composition

wherein the composition comprises at least one compound having a partial structure represented by formula (1) defined in [1].

[21] A polymer comprising at least one repeating unit represented by formula (2):

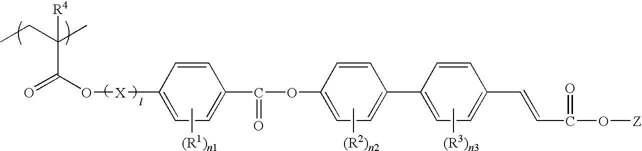

where, each of R¹, R² and R³ independently represents a substituent; R⁴ represents a hydrogen atom or substituent; X represents a divalent linking group selected from Linking Group I shown below, or a divalent linking group formed by combining two or more species selected from Linking Group I shown below; Z represents a substituted or non-substituted alkyl group or aryl group; each of n1, n2 and n3 represents an integer of 0 to 4; and each of l, m and n represents an integer of 0 to 4;

### Linking Group I:

single bond, —O—, —CO—, —NR⁶— (R⁶ represents a hydrogen atom, alkyl group or aryl group), —S—, —SO₂—, —P(═O)(OR⁷)— (R⁷ represents an alkyl group or aryl group), alkylene group and arylene group.

[22] The polymer as set forth in [21], further comprising a repeating unit represented by formula (5) and/or formula (7) below:

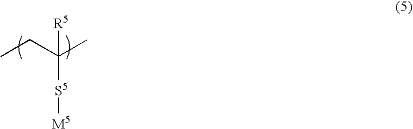

where, R⁵ represents a hydrogen atom or substituent, S⁵ represents a divalent linking group, and M⁵ represents a mesogen group;

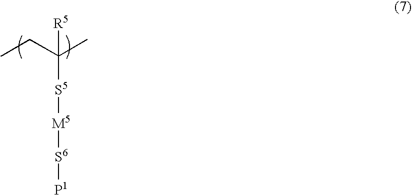

where, R⁵ represents a hydrogen atom or substituent, S⁵ represents a divalent linking group, M⁵ represents a mesogen group, S⁶ represents a divalent linking group, and P¹ represents a polymerizable group.

### BRIEF DESCRIPTION OF THE DRAWINGS

FIG. 1 is a rough schematic drawing showing one example of a flow of the producing process of the present invention.

FIG. 2 is a rough cross-sectional drawing of one example of a substrate employed in the liquid crystal display device of the invention.

FIG. 3 is a rough cross-sectional drawing of one example of the liquid crystal display device of the invention.

In the drawings, reference numerals have following meanings.
11 transparent substrate
12 black matrix (barrier wall)
13 optically anisotropic layer (optically anisotropic film of the invention)
14 color filter layer
21 substrate
22 black matrix (barrier wall)
23 color filter layer
24 second optically anisotropic layer
25 transparent electrode layer
26 alignment layer
27 patterned optically anisotropic layer (optically anisotropic film of the invention)
31 liquid crystal
32 TFT
33 polarizing layer
34 cellulose acetate film (polarizing plate-protective film)
35 cellulose acetate film, or optical compensatory sheet
36 polarizing plate
37 liquid crystal cell

### DETAILED DESCRIPTION OF THE INVENTION

The invention will be described in detail below. The expression “from a lower value to an upper value” referred herein means that the range intended by the expression includes both the lower value and the upper value.

In this description, the term “polymer” is used for not only any homopolymers composed of a single species of monomers, but also any so-called copolymers composed of two or more species of monomers. In this specification, “group” such as alkyl group or the like may have substituent(s) or no substituent, unless otherwise specifically noted. Accordingly, an exemplary phrase of “an alkyl group having A to B carbon atoms” means that the alkyl group may have substituent(s) or not. As for the alkyl group having substituent(s), it is to be understood that carbon atoms contained in the substituent(s) are included in the number of A and B.

In the description, Re(λ) and Rth(λ) each indicate a retardation in plane (unit:nm) and a retardation along thickness direction (unit:nm) at a wavelength λ. Re(λ) is measured by applying a light having a wavelength of λ nm in the normal line direction of a sample such as a film, using KOBRA-21ADH or WR (by Oji Scientific Instruments). Selection of wavelength for measuring may be performed by manual change of a wavelength-selection filter or by programming conversion of measured data.

When the sample to be tested is represented by an uniaxial or biaxial refractive index ellipsoid, then its Rth(λ) is calculated according to the method mentioned below.

With the in-plane slow axis (determined by KOBRA 21ADH or WR) taken as the inclination axis (rotation axis) of the sample (in case where the sample has no slow axis, the rotation axis of the sample may be in any in-plane direction of the sample), Re(λ) of the sample is measured at 6 points in all thereof, up to +50° relative to the normal line direction of the sample at intervals of 10°, by applying a light having a wavelength of λ nm from the inclined direction of the sample.

With the in-plane slow axis from the normal line direction taken as the rotation axis thereof, when the sample has a zero retardation value at a certain inclination angle, then the symbol of the retardation value of the sample at an inclination angle larger than that inclination angle is changed to a negative one, and then applied to KOBRA 21ADH or WR for computation.

With the slow axis taken as the inclination axis (rotation axis) (in case where the sample has no slow axis, the rotation axis of the sample may be in any in-plane direction of the film), the retardation values of the sample are measured in any inclined two directions; and based on the data and the mean refractive index and the inputted thickness of the sample, Rth may be calculated according to the following formulas (11) and (12):

$$\begin{matrix}Re ( θ ) = [ nx - \frac{ny \times nz}{\sqrt{\begin{matrix}{\{ ny sin ( {sin}^{- 1} ( \frac{sin ( - θ )}{nx} ) ) \}}^{2} + \\ {\{ nz cos ( {sin}^{- 1} ( \frac{sin ( - θ )}{nx} ) ) \}}^{2}\end{matrix}}} ] \times \frac{d}{cos \{ {sin}^{- 1} ( \frac{sin ( - θ )}{nx} ) \}} \\ & ( 11 ) \\ Rth = \{ ( nx + ny ) / 2 - nz \} \times d & ( 12 )\end{matrix}$$

wherein Re(θ) means the retardation value of the sample in the direction inclined by an angle θ from the normal line direction; nx means the in-plane refractive index of the sample in the slow axis direction; ny means the in-plane refractive index of the sample in the direction vertical to nx; nz means the refractive index of the sample vertical to nx and ny; and d is a thickness of the sample.

When the sample to be tested can not be represented by a monoaxial or biaxial index ellipsoid, or that is, when the sample does not have an optical axis, then its Rth(λ) may be calculated according to the method mentioned below.

With the in-plane slow axis (determined by KOBRA 21ADH or WR) taken as the inclination axis (rotation axis) of the sample, Re(λ) of the sample is measured at 11 points in all thereof, from −50° to +50° relative to the normal line direction of the sample at intervals of 10°, by applying a light having a wavelength of λ nm from the inclined direction of the sample. Based on the thus-determined retardation data of Re(λ), the mean refractive index and the inputted thickness of the sample, Rth(λ) of the sample is calculated with KOBRA 21ADH or WR.

The mean refractive index may be used values described in catalogs for various types of optical films. When the mean refractive index has not known, it may be measured with Abbe refractometer. The mean refractive index for major optical film is described below: cellulose acetate (1.48), cycloolefin polymer (1.52), polycarbonate (1.59), polymethylmethacrylate (1.49), polystyrene (1.59).

The mean refractive index and the film thickness are inputted in KOBRA 21ADH or WR, nx, ny and nz are calculated therewith. From the thus-calculated data of nx, ny and nz, Nz=(nx−nz)/(nx−ny) is further calculated.

In the description, the sign of Rth is defined as follows. If the value of retardation, measured for light of 550 nm entering the film in the direction rotated +20° relative to the normal line of the film, while assuming the in-plane slow axis as an axis of inclination (axis of rotation), is more than Re, the sign of Rth is assumed positive; if the value of retardation is less than Re, the sign of Rth is assumed negative. Exceptionally, regarding a sample film having |Rth/Re| values of 9 or larger, the sign of Rth is defined as follows. Using a polarization microscope with a freely-rotatable stage, the sample film is observed in the direction rotated +40° relative to the normal line of the film while assuming the in-plane fast axis as an axis of inclination (axis of rotation). Then the slow axis of the sample film is determined using a polarizer plate as a test plate, and if the slow axis determined in this way lies in parallel with the film surface, the sign of Rth is assumed positive; and if the slow axis determined in this way lies in the thickness direction of the film, the sign of Rth is assumed negative.

In the description, λ represents 611±5 nm, 545±5 nm and 435±5 nm respectively for R, G and B, and represents 545±5 nm or 590±5 nm unless otherwise colors are specifically noted.

In this specification, an expression of “substantially” in relation to angle means that measured angle falls in an error range of smaller than ±5° with respect to the strict angle. The error range with respect to the strict angle may preferably be smaller than 4°, and more preferably be smaller than 3°. An expression “substantially” in relation to retardation means that measured retardation falls in an error range of smaller than ±5% with respect to the strict angle. A phrase “Re is not zero” means that Re is 5 nm or larger. Unless otherwise specifically noted, wavelength under which refractive index is measured is 550 nm. In this specification, the “visible light” means light having wavelength ranging from 400 to 700 nm.

### [Optically Anisotropic Film]

The present invention relates to an optically anisotropic film comprising at least one compound having a partial structure represented by formula (1) below. The partial structure shown below aligns when irradiated with polarized light, and expresses birefringence. Therefore, an optically anisotropic film showing desired optical characteristics may be formed without using an alignment film, and for example a fine optically anisotropic film may be formed without using a technique such as patterning.

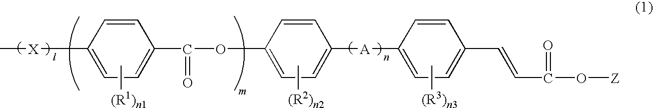

In the formula, each of R¹, R² and R³ independently represents a substituent; X represents a divalent linking group selected from Linking Group I shown below, or a divalent linking group formed by combining two or more species selected from Linking Group I shown below; A represents —COO—, —OCO—, or substituted or non-substituted phenylene group, oxadiazole group or alkynylene group; Z represents a substituted or non-substituted alkyl group or aryl group; each of n1, n2 and n3 independently represents an integer of 0 to 4; and each of m and n represents an integer of to 4.

### Linking Group I

Single bond, —O—, —CO—, —NR⁶— (R⁶ represents a hydrogen atom, alkyl group or aryl group), —S—, —SO₂—, —P(═O)(OR⁷)— (R⁷ represents an alkyl group or aryl group), alkylene group and arylene group.

Examples of the substituent represented by R¹, R² or R³ include alkyls (preferably C₁₋₂₀, more preferably C₁₋₁₂, and even more preferably C₁₋₈ alkyls) such as methyl, ethyl, isopropyl, tert-butyl, n-octyl, n-decyl, n-hexadecyl, cyclopropyl, cyclopentyl and cyclohexyl; alkenyls (preferably C₂₋₂₀, more preferably C₂₋₁₂, and even more preferably C₂₋₈ alkenyls) such as vinyl, allyl, 2-butenyl and 3-pentenyl; alkynyls (preferably C₂₋₂₀, more preferably C₂₋₁₂, and even more preferably C₂₋₈ alkynyl) such as propargyl and 3-pentynyl; aryls (preferably C₆₋₃₀, more preferably C₆₋₂₀, and even more preferably C₆₋₁₂ aryls) such as phenyl, p-methyl phenyl and naphthyl; substituted or non-substituted aminos (preferably C₀₋₂₀, more preferably C₀₋₁₀, and even more preferably C₀₋₆ aminos) such as non-substituted amino, methylamino, ethylamino, dimethylamino, diethylamino and anilino; alkoxys (preferably C₁₋₂₀ alkoxys) such as methoxy, ethoxy and butoxy; alkoxycarbonyls (preferably C₂₋₂₀, more preferably C₂₋₁₂, and even more preferably C₂₋₈ alkoxycarbonyl) such as methoxycarbonyl and ethoxycarbonyl; acyloxys (preferably C₂₋₂₀, more preferably C₂₋₁₆, and even more preferably C₂₋₁₀ acyloxys) such as acetoxy and benzoyloxy; acylaminos (preferably C₂₋₂₀, more preferably C₂₋₁₆, and even more preferably C₂₋₁₀ acylaminos) such as acetylamino and benzoylamino; alkoxycarbonylaminos (preferably C₂₋₂₀, more preferably C₂₋₁₆, and even more preferably C₂₋₁₀ alkoxycarbonylaminos) such as methoxycarbonylamino; aryloxycarbonylaminos (preferably C₇₋₂₀, more preferably C₇₋₁₆, and even more preferably C₇₋₁₂ aryloxycarbonylaminos) phenyloxycarbonylamino; sulfonylaminos (preferably C₁₋₂₀, more preferably C₁₋₁₆, and even more preferably C₁₋₁₂ sulfonylaminos) such as methane sulfonylamino and benzene sulfonylamino; sulfamoyls (preferably C₀₋₂₀, more preferably C₀₋₁₆, and even more preferably C₀₋₁₂ sulfamoyls) such as non-substituted sulfamoyl, methyl sulfamoyl, dimethyl sulfamoyl and phenyl sulfamoyl; carbamoyls (preferably C₁₋₂₀, more preferably C₁₋₁₆, and even more preferably C₁₋₁₂ carbamoyls) such as non-substituted carbamoyl, methyl carbamoyl, diethyl carbamoyl and phenyl carbamoyl; alkylthios (preferably C₁₋₂₀, more preferably C₁₋₁₆, and even more preferably C₁₋₁₂ alkylthios) such as methylthio and ethylthio; arylthios (preferably C₆₋₂₀, more preferably C₆₋₁₆, and even more preferably C₆₋₁₂ arylthios) such as phenylthio; sulfonyls (preferably C₁₋₂₀, more preferably C₁₋₁₆, and even more preferably C₁₋₂ sulfonyls) such as mesyl and tosyl; sulfinyls (preferably C₁₋₂₀, more preferably C₁₋₁₆, and even more preferably C₁₋₁₂ sulfinyls) such as methane sulfinyl and benzene sulfinyl; ureido groups (preferably C₁₋₂₀, more preferably C₁₋₁₆, and even more preferably C₁₋₁₂ ureido groups) such as non-substituted ureido, methylureido and phenylureiod; amide phosphate groups (preferably C₁₋₂₀, more preferably C₁₋₁₆, and even more preferably C₁₋₁₂ amide phosphate groups) such as diethylphosphoramide and phenylphosphoramide; hydroxy, mercapto, halogen atoms such as fluorine, chlorine, bromine and iodine atoms; cyano, sulfo, carboxyl, nitro, hydroxamic acid group, sulfino, hydrazino, imino, heterocyclic groups (preferably C₁₋₃₀, and more preferably C₁₋₁₂ heterocyclic groups in which at least one hetero atoms such as nitrogen, oxygen or sulfur atoms is embedded) such as imidazolyl, pyridyl, quinolyl, furyl, piperidyl, morpholino, benzoxazolyl, benzoimidazolyl and benzothiazolyl; silyl groups (preferably C₃₋₄₀, more preferably C₃₋₃₀, and even more preferably C₃₋₂₄ silyl groups) such as trimethyl silyl and triphenyl silyl.

These exemplified substituents may have at least one substituent. When there are two or more substituents, they may be same or different from each other. And they may bind to each other to form a ring.

The substituent represented by each of R¹, R² and R³ is preferably an alkyl group, aryl group, alkoxy group, alkoxycarbonyl group, acyloxy group, acylamino group, sulfonylamino group, alkylthio group, or halogen atom, more preferably is an alkyl group, aryl group, alkoxy group, alkoxycarbonyl group, acyloxy group, or halogen atom, and still more preferably is an alkyl group, alkoxy group, or halogen atom. R¹, R² and R³ preferably represent an alkyl group, alkoxy group, or halogen atom.

Each of n1, n2 and n3 is preferably an integer of 0 to 3, and more preferably an integer of 0 to 2. More specifically, the compounds wherein any of R¹, R² and R³ is absent (n1, n2 or n3 is 0) are preferable, as well as the compounds wherein R¹, R² and R³ are present and each represent an alkyl group, alkoxy group, or halogen atom.

X preferably has —O—, —CO—, —NR⁶—, alkylene group, or arylene group therein, more preferably has —O—, —CO—, —NR⁶—, or alkylene group therein, and still more preferably has —O—, —CO—, or alkylene group therein. Regarding X having an alkylene group therein, the alkylene group preferably has 1 to 10 carbon atoms, more preferably 1 to 8, and particularly preferably 1 to 6. Particularly preferable examples of the alkylene group include methylene, ethylene, trimethylene, tetrabutylene, and hexamethylene groups. Regarding X having an arylene group therein, the arylene group preferably has 6 to 24 carbon atoms, more preferably 6 to 18, and particularly preferably 6 to 12. Particularly preferable examples of the arylene group include phenylene and naphthalene groups. Regarding X having a divalent linking group obtained by combining an alkylene group and an arylene group (that is, an aralkylene group) therein, the aralkylene group preferably has 7 to 34 carbon atoms, more preferably 7 to 26, and particularly preferably 7 to 16. Particularly preferable examples of the aralkylene group include phenylenemethylene, phenyleneethylene, and methylenephenylene groups. The groups exemplified as X may have appropriate substituent(s).

In the formula, “l” represents an integer of 0 to 4, and preferably 0 or 1.

In the formula, “A” represents —COO—, —OCO—, phenylene group, oxadiazole group, or alkynylene group.

In the formula, “n” represents an integer of 0 to 4, preferably 0 or 1. The compounds in which wherein n is 0 has a biphenyl structure having two benzene rings linked via a single bond as a partial structure. The phenylene group may have substituent(s), wherein examples of the substituent(s) may be those exemplified for R¹, R² and R³. The same will apply also to the preferable examples.

In the formula, Z represents a substituted or non-substituted, alkyl group or aryl group. The alkyl group represented by Z preferably has 1 to 10 carbon atoms, more preferably 1 to 8, and especially preferably 1 to 6. The alkyl group may be branched or cyclic. The aryl group represented by Z preferably has 6 to 24 carbon atoms, more preferably 6 to 18, and especially preferably 6 to 12. Especially preferable examples of the aryl group include phenyl group and naphthalene group. Z is preferably an alkyl group. The alkyl group and the aryl group represented by Z may have substituent(s), wherein examples of the substituent includes those exemplified for R¹, R² and R³. Regarding Z being the alkyl group or the aryl group having the substituent (s), the substituent may contain a polymerizable group. Presence of the polymerizable group is preferable because the film may be made harder, and fluctuation in the optical characteristics may more effectively be reduced. The compound may include two or more polymerizable groups. For example, Z at one end may contain a polymerizable group, and also X at the other end may contain a polymerizable group.

The polymerizable group is not specifically limited, wherein any polymerizable group capable of proceeding addition polymerization (including ring-opening polymerization) or condensation polymerization may be preferable. Examples of the polymerizable group are shown below:

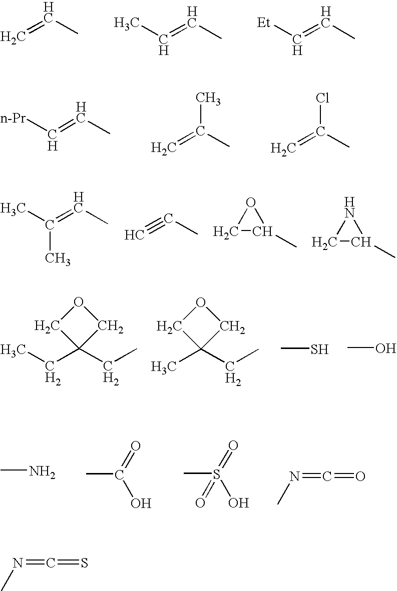

The polymerizable group is preferably any polymerizable group capable of proceeding radical polymerization or cationic polymerization. Radical polymerizable group usable herein may be any of those, wherein (meth)acrylate group may be exemplified as a preferable example. Cationic polymerizable group usable herein may be any of those, wherein alicyclic ether group, cyclic acetal group, cyclic lactone group, cyclic thioether group, spiro-orthoester group, and vinyloxy group may be exemplified as preferable example. Among these, alicyclic ether group and vinyloxy group are preferable, and epoxy group, oxetanyl group and vinyloxy group are particularly preferable. As has been described in the above, the compound may has two or more species of polymerizable group. Among the compounds having two or more species of polymerizable group, the compounds, having polymerizable groups capable of polymerizing according to a different polymerization mechanism from each other, are preferable. Preferable examples of such a compound include a compound having two polymerizable group, one of which is a radial polymerizable group, and another of which is a cationic polymerizable group.

In the formula, “m” represents an integer of 0 to 4, and preferably 0 or 1.

Specific examples of the compounds having structures represented by formula (1) include, but are not limited to, those shown below.

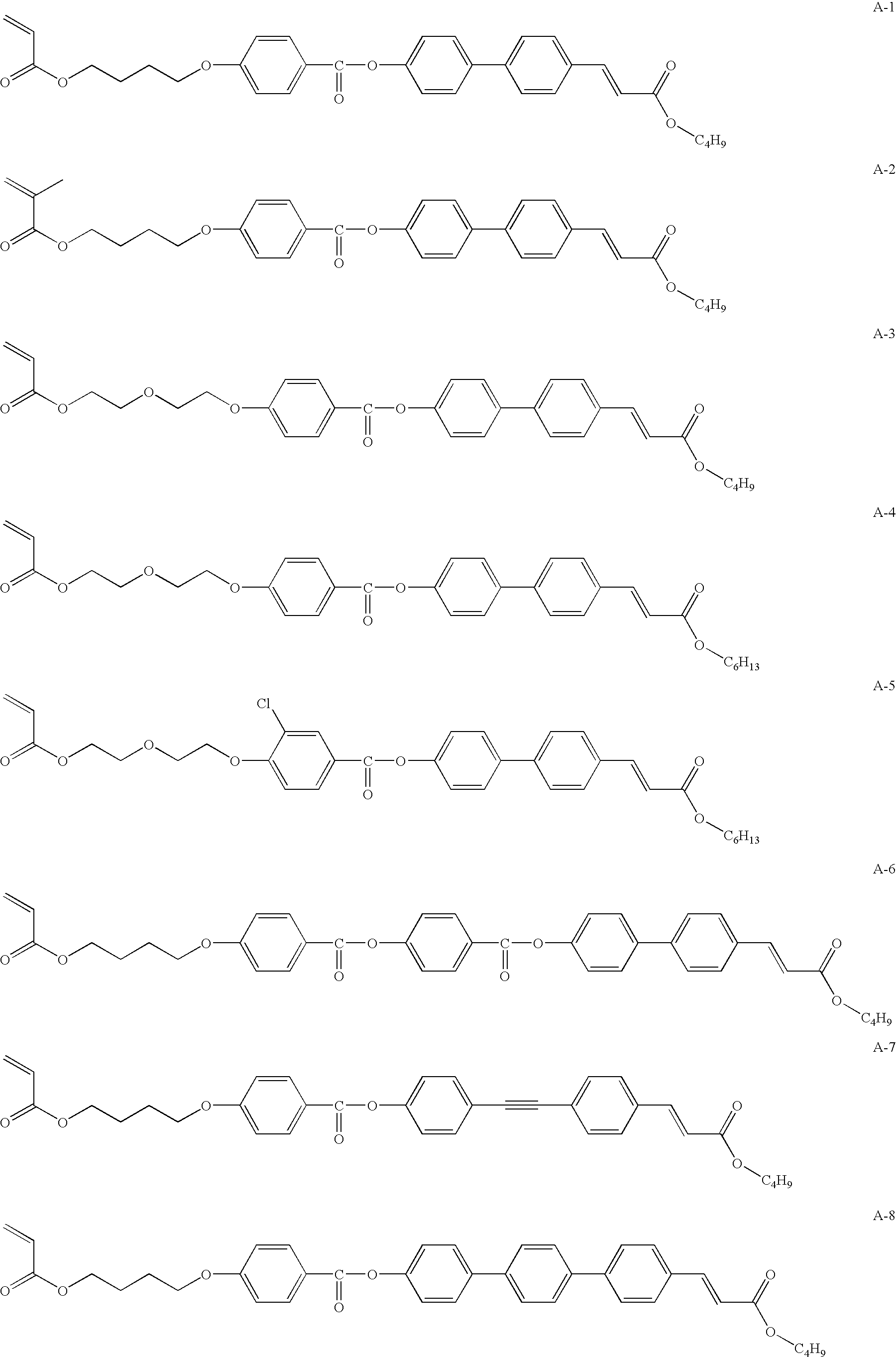

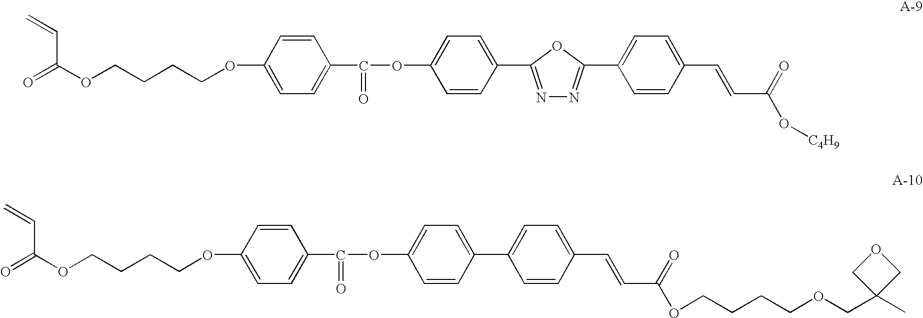

The optically anisotropic film of the present invention may be formed by using polymerizable monomers represented by A-1 to A-10 without modification, or by using homopolymers obtained by polymerizing a single species of these monomers, or by using copolymers obtained by copolymerizing different species of these monomers. For example, the optically anisotropic film is formed using any monomer having two species of polymerizable group, such as A-10 having a radical polymerizable group as one of the polymerizable group and having a cationic polymerizable group as another polymerizable group. Such monomer may be polymerized at the polymerizable group thereof not contained in the partial structure of formula (1) (which corresponds to the radical polymerizable group of Compound A-10), the obtained polymer may be irradiated with polarized light so as to align the partial structure of formula (1), and the polymer may further be polymerized at the other polymerizable group (which corresponds to the cationic polymerizable group of Compound A-10). The process is desirable in terms of obtaining an optically anisotropic film more improved in the durability.

One example of the compounds used for forming the optically anisotropic film of the present invention is a polymer compound having the partial structure represented by formula (1) in the side chain(s) thereof, and more specifically, a polymer containing a repeating unit having the partial structure represented by formula (1). More preferably, the compound is a polymer having a repeating unit represented by formula (1)′ below, and still more preferably a polymer having a repeating unit represented by formula (2) below:

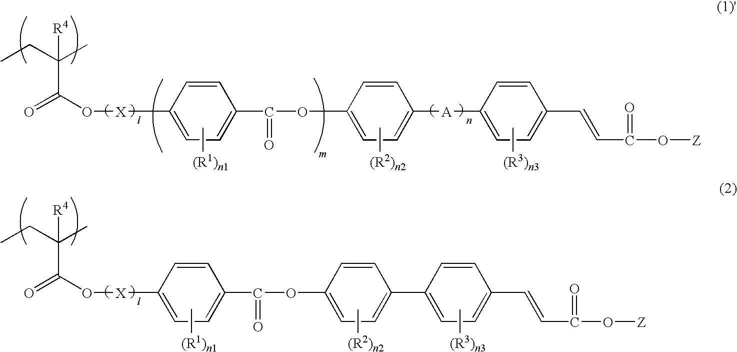

In the formulas, R⁴ represents a hydrogen atom or substituent, and other symbols are used for the same meaning with those in formula (1), with the same preferable ranges.

Examples of the substituent represented by R⁴ may be same as those exemplified as the substituents represented by R¹, R² and R³. R⁴ is preferably a hydrogen atom or alkyl group, and more preferably a hydrogen atom or methyl group.

The polymer may be composed of only a single species of, or two or more species of the repeating unit represented by formula (1)′ or (2). The polymer may have one species of, or two or more species of repeating unit other than the repeating unit represented by formula (1)′ or (2). The above-described other repeating unit is not specifically limited, and is preferably selected from the repeating units derived from various monomers capable of proceeding radical polymerization reaction.

Examples of the monomer from which other repeating unit is derived include those shown below.

(1) Alkenes:

ethylene, propylene, 1-buten, isobuten, 1-hexene, 1-dodecene, 1-octadecene, 1-eicocene, hexafluoropropene, vinylidene fluoride, chlorotrifluoroethylene, 3,3,3-trifuluoropropylene, tetrafluoroethylene, vinyl chloride, vinylidene chloride or the like;

(2) Dienes:

1,3-butadinene, isoprene, 1,3-pentadiene, 2-ethyl-1,3-butadiene, 2-n-propyl-1,3-butadiene, 2,3-dimethyl-1,3-butadiene, 2-methyl-1,3-pentadiene, 1-phenyl-1,3-butadiene, 1-α-naphtyl-1,3-butadiene, 1-β-naphtyl-1,3-butadiene, 2-chloro-1,3-butadiene, 1-bromo-1,3-butadiene, 1-chlorobutadiene, 2-fluoro-1,3-butadiene, 2,3-dichloro-1,3-butadiene, 1,1,2-trichloro-1,3-butadiene, 2-cyano-1,3-butadiene, 1,4-divinyl cyclohexane or the like;

(3) α,β-unsaturated carboxylic acid derivatives:

(3a) Alkyl acrylates:

methyl methacrylate, ethyl acrylate, n-propyl acrylate, isopropyl acrylate, n-butyl acrylate, isobutyl acrylate, sec-butyl acrylate, tert-butyl acrylate, amyl acrylate, n-hexyl acrylate, cyclohexyl acrylate, 2-ethylhexyl acrylate, n-octyl acrylate, tert-octyl acrylate, dodecyl acrylate, phenyl acrylate, benzyl acrylate, 2-chloroethyl acrylate, 2-bromoethyl acrylate, 4-chlorobutyl acrylate, 2-cyanoethyl acrylate, 2-acetoxyethyl acrylate, methoxybenzyl acrylate, 2-chlorocyclohexyl acrylate, furfuryl acrylate, tetrahydrofurfuryl acrylate, 2-methoxyethyl acrylate, ω-methoxy polyethyleneglycol acrylate (having additional molar number, n, of 2 to 100), 3-metoxybutyl acrylate, 2-ethoxyethyl acrylate, 2-butoxyethyl acrylate, 2-(2-butoxyethoxy)ethyl acrylate, 1-bromo-2-methoxyethyl acrylate, 1,1-dichloro-2-ethoxyethyl acrylate, glycidyl acrylate or the like;

(3b) Alkyl methacrylates:

methyl methacrylate, ethyl methacrylate, n-propyl methacrylate, isopropyl methacrylate, n-butyl methacrylate, isobutyl methacrylate, sec-butyl methacrylate, tert-butyl methacrylate, amyl methacrylate, n-hexyl methacrylate, cyclohexyl methacrylate, 2-ethylhexyl methacrylate, n-octyl methacrylate, stearyl methacrylate, benzyl methacrylate, phenyl methacrylate, allyl methacrylate, furfuryl methacrylate, tetarahydrofurfuryl methacrylate, crezyl methacrylate, naphthyl methacrylate, 2-methoxyethyl methacrylate, 3-methoxybutyl methacrylate, ω-methoxypolyethyleneglycol methacrylate (having additional molar number, n, of 2 to 100), 2-acetoxyethyl methacrylate, 2-ethoxyethyl methacrylate, 2-butoxyethyl methacrylate, 2-(2-butoxyethoxy)ethyl methacrylate, glycidyl methacrylate, 3-trimetoxysilylpropyl methacrylate, allyl methacrylate, 2-isosyanate ethyl methacrylate or the like;

(3c) Diesters of unsaturated polycarboxylic acids:

dimethyl maleate, dibutyl maleate, dimethyl itaconate, dibutyl itaconate, dibutyl crotonate, dihexyl crotonate, diethyl fumarate, dimethyl fumarate or the like;

(3d) Amides of α,β-unsaturated carboxylic acids:

N,N-dimethyl acrylamide, N,N-diethyl acrylamide, N-n-propyl acrylamide, N-tert-butyl acrylamide, N-tert-octyl acrylamide, N-cyclohexyl acrylamide, N-phenyl acrylamide, N-(2-acetoacetoxyethyl)acrylamide, N-benzyl acrylamide, N-acryloyl morpholine, diacetone acrylamide, N-methyl maleimide or the like;

(4) Unsaturated nitriles:

acrylonitrile, methacrylonitrile or the like;

(5) Styrene or derivatives thereof:

styrene, vinyltoluene, ethylstyrene, p-tert-butylstyrene, p-vinyl methyl benzoate, α-methyl styrene, p-chloromethyl styrene, vinyl naphthalene, p-methoxy styrene, p-hydroxy methyl styrene, p-acetoxy styrene or the like;

(6) Vinyl esters:

vinyl acetate, vinyl propanate, vinyl butyrate, vinyl isobutyrate, vinyl benzoate, vinyl salicylate, vinyl chloroacetate, vinyl methoxy acetate, vinyl phenyl acetate or the like;

(7) Vinyl ethers:

methyl vinyl ether, ethyl vinyl ether, n-propyl vinyl ether, isopropyl vinyl ether, n-butyl vinyl ether, isobutyl vinyl ether, tert-butyl vinyl ether, n-pentyl vinyl ether, n-hexyl vinyl ether, n-octyl vinyl ether, n-dodecyl vinyl ether, n-eicosyl vinyl ether, 2-ethylhexyl vinyl ether, cyclohexyl vinyl ether, fluorobutyl vinyl ether, fluorobutoxyethyl vinyl ether or the like; and

(8) Other monomers

N-vinyl pyrrolidone, methyl vinyl ketone, phenyl vinyl ketone, methoxy ethyl vinyl ketone, 2-vinyl oxazoline, 2-isopropenyl oxazoline or the like.

Particularly preferably, the other repeating unit is a unit represented by formula (5):

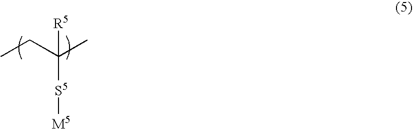

In the formula, R⁵ represents a hydrogen atom or substituent, S⁵ represents a divalent linking group, and M⁵ represents a mesogen group.

Examples of the substituent represented by R⁵ include those exemplified as the substituents represented by R¹ or the like in formula (1). Among these, alkyl group or halogen atom is preferable.

R⁵ is preferably a hydrogen atom, alkyl group having 1 to 6 carbon atoms, or chlorine atom, more preferably a hydrogen atom, methyl group, ethyl group, or chlorine atom, and still more preferably a hydrogen atom or methyl group.

S⁵ is preferably a divalent linking group selected from the group consisting of alkylene group, alkenylene group, arylene group, divalent hetero ring residue, —CO—, —NR¹⁵— (R¹⁵ represents an alkyl group having 1 to 6 carbon atoms, or hydrogen atom), —O—, —S—, —SO—, —SO₂— and combinations of them. The alkylene group preferably has 1 to 12 carbon atoms. The alkenylene group preferably has 2 to 12 carbon atoms. The arylene group preferably has 6 to 10 carbon atoms. The alkylene group, alkenylene group and arylene group may be substituted, if possible, by a substituent (alkyl group, halogen atom, cyano group, alkoxy group, acyloxy group, etc.), but is preferably non-substituted.

S⁵ preferably has —O—, —CO—, —NR¹⁵— (R¹⁵ represents an alkyl group or hydrogen atom having 1 to 6 carbon atoms), alkylene group or arylene group therein, and particularly preferably has —O—, —CO—, alkylene group or arylene group therein. Still also preferably, S⁵ is composed only of —O—, —CO—, alkylene group or arylene group.

The mesogen group represented by M⁵ adoptable herein may be those described in Makromol. Chem., Vol. 190, p. 2255 (1989), and Advanced Materials Vol. 5, p. 107 (1993) and so forth.

The mesogen group represented by formula (6) below is more preferable:

-Cy¹-L¹-(Cy²-L²)ₚ-Cy³ (6)

In the formula, each of L¹ and L² independently represents a single bond or divalent linking group, each of Cy¹, Cy² and Cy³ independently represents a divalent cyclic group, and p represents an integer of 0 to 2. When p is 2, two “L²”s may be same or different from each other, and also two “Cy²”s may be same or different from each other.

In formula (6), each of L¹ and L² independently represents a divalent linking group selected from the set consisting of —O—, —S—, —CO—, —NR¹⁶—, divalent chain-like group, divalent cyclic group and combinations of them, or, a single bond. R¹⁶ represents an alkyl group having 1 to 7 carbon atoms or hydrogen atom, and is preferably an alkyl group having 1 to 4 carbon atoms, or hydrogen atom, more preferably a methyl group, ethyl group or hydrogen atom, and most preferably a hydrogen atom.

The divalent chain-like group is preferably an alkylene group, alkenylene group or alkynylene group, all of which may have substituent(s). The substituent is preferably a halogen atom. The divalent chain-like group is preferably an alkylene group or alkenylene group, and more preferably a non-substituted alkylene group or non-substituted alkenylene group. The alkylene group may be branched. The alkylene group preferably has 1 to 12, more preferably 2 to 10, and still more preferably 2 to 8 carbon atoms. The alkenylene group may be branched. The alkenylene group preferably has 2 to 12, more preferably 2 to 10, and still more preferably 2 to 8 carbon atoms. The alkynylene group may be branched. The alkynylene group preferably has 2 to 12, more preferably 2 to 10, and still more preferably 2 to 8 carbon atoms.

Specific examples of the divalent chain-like group include ethylene group, trimethylene group, propylene group, tetramethylene group, 2-methyl-tetramethylene group, pentamethylene group, hexamethylene group, octamethylene group, 2-butenylene group, and 2-butynylene group.

The divalent cyclic group has the same meaning with Cy¹, Cy² and Cy³ described later, with the same preferable ranges.

In formula (6), p is preferably an integer of 0 or 1.

In formula (6), each of Cy¹, Cy² and Cy³ independently represents a divalent cyclic group. Ring contained in the cyclic group may preferably be a five-membered ring, six-membered ring or seven-membered ring, more preferably a five-membered ring or six-membered ring, and still more preferably a six-membered ring. Ring contained in the cyclic group may be a monocycle or may be a condensed ring, wherein the monocycle is more preferable. Ring contained in the cyclic group may be any of aromatic ring, alicyclic group and hetero ring. Examples of the aromatic ring include benzene ring and naphthalene ring. Examples of the alicyclic group include cyclohexane ring. Examples of the hetero ring include pyridine ring and pyrimidine ring. The cyclic group having a benzene ring is preferably 1,4-phenylene group. The cyclic group having a naphthalene ring is preferably naphthalene-1,5-diyl group or naphthalene-2,6-diyl group. The cyclic group having a cyclohexane ring is preferably 1,4-cyclohexylene group. The cyclic group having a pyridine ring is preferably pyridine-2,5-diyl group. The cyclic group having a pyrimidine ring is preferably pyrimidine-2,5-diyl group.

The cyclic group may have substituent(s). Examples of the substituent include halogen atom, cyano group, nitro group, alkyl group having 1 to 5 carbon atoms, alkyl group substituted by halogen atom and having 1 to 5 carbon atoms, alkoxy group having 1 to 5 carbon atoms, alkylthio group having 1 to 5 carbon atoms, acyloxy group having 2 to 6 carbon atoms, alkoxycarbonyl group having 2 to 6 carbon atoms, carbamoyl group, carbamoyl group substituted by alkyl group having 2 to 6 carbon atoms, and acylamino group having 2 to 6 carbon atoms.

Among the repeating units represented by formula (5), a repeating unit represented by formula (7) below is preferable:

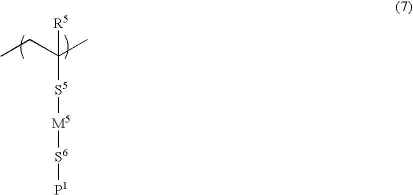

In the formula, any symbols same as those in formula (5) in the above are used for the same meaning, with the same preferable ranges. S⁶ represents a divalent linking group, and has the same meaning with S⁵ in formula (5), with the same preferable ranges. P¹ represents a polymerizable group. Examples of the polymerizable group represented by P¹ include those exemplified for the polymerizable group contained in Z of formula (1), where also the preferable ranges are same therewith.

Specific examples of monomers from which other repeating units are derived include, but are not limited to, those shown below.

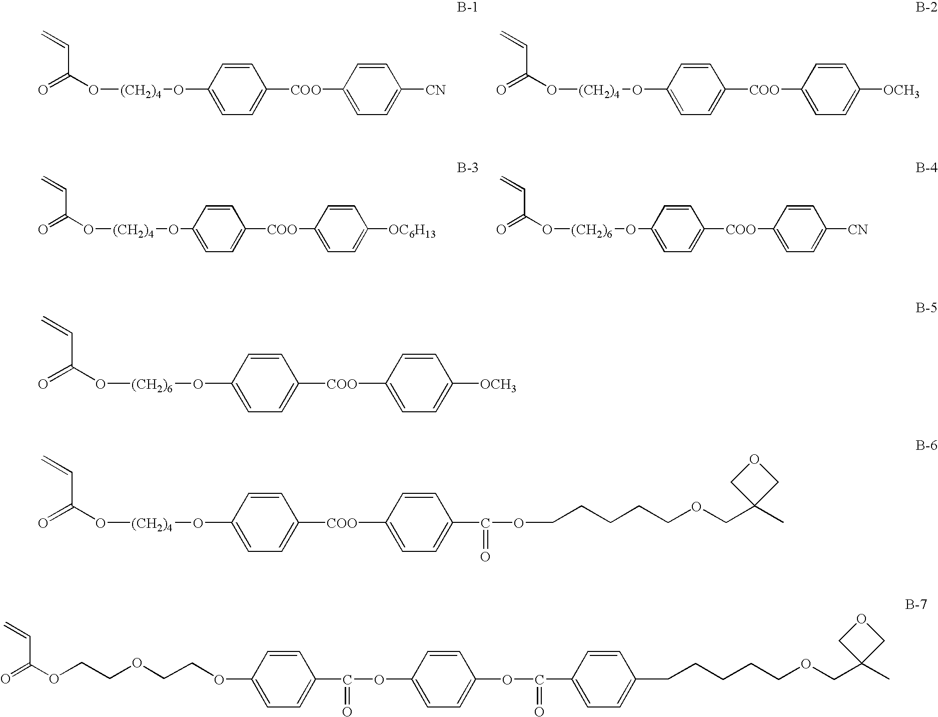

In the polymer, the repeating unit having the partial structure represented by formula (1) is preferably contained to as much as 3 mol % or more, more preferably 5 mol % or more, and still more preferably 10 mol % of the total amount of the repeating unit. Although, of course, content of the repeating unit may be 100 mol %, the polymer preferably contains other repeating unit in view of expression performance of optical anisotropy, and more specifically, the other repeating unit is preferably contained to as much as 10 to 97 mol % or around.

In particular, for the purpose of improving Nz value described later, it may be good enough to raise the molar content of the repeating unit having the partial structure represented by formula (1). The polymer used for forming the optically anisotropic film having an Nz value of Nz 1.1 to 7.0 preferably contains the repeating unit having the partial structure represented by formula (1) to as much as 16 to 75 mol %.

Specific examples of the polymer comprising the repeating unit having the partial structure represented by formula (1) (the repeating units represented by the formulae (1)′ and (2)) include, but are not limited to, those shown below. Numerals given in the formulas represent molar percentage of the individual repeating unit.

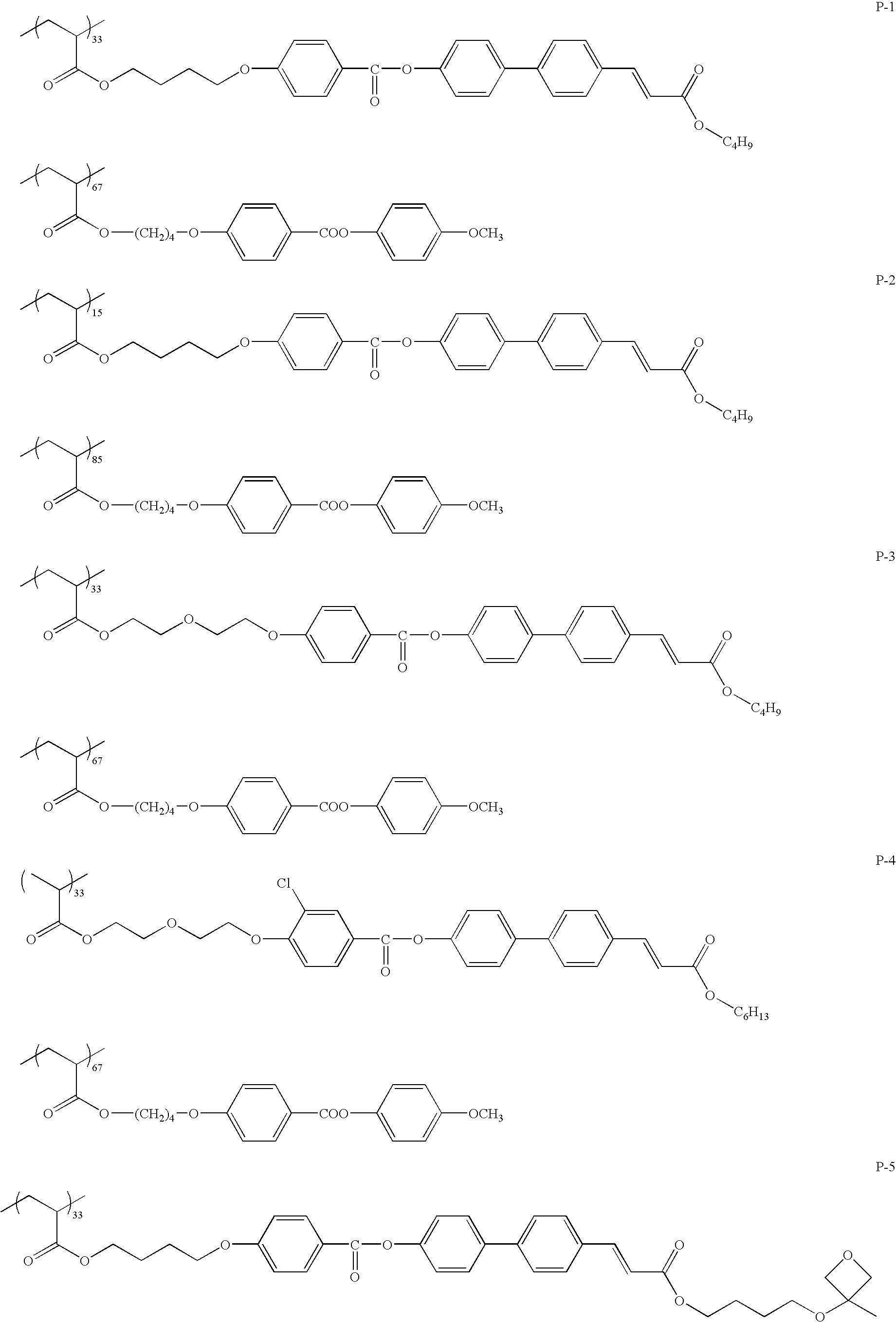

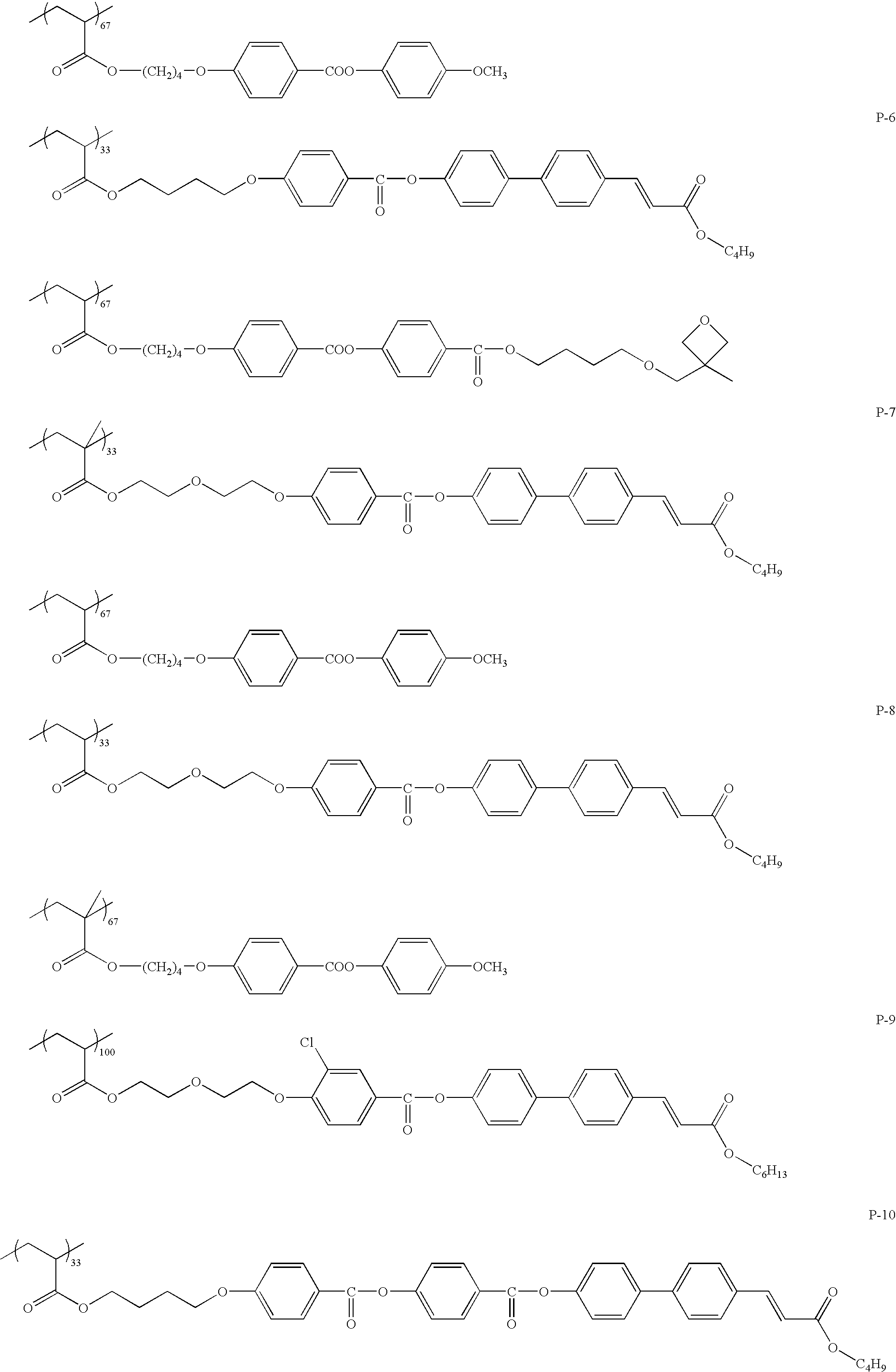

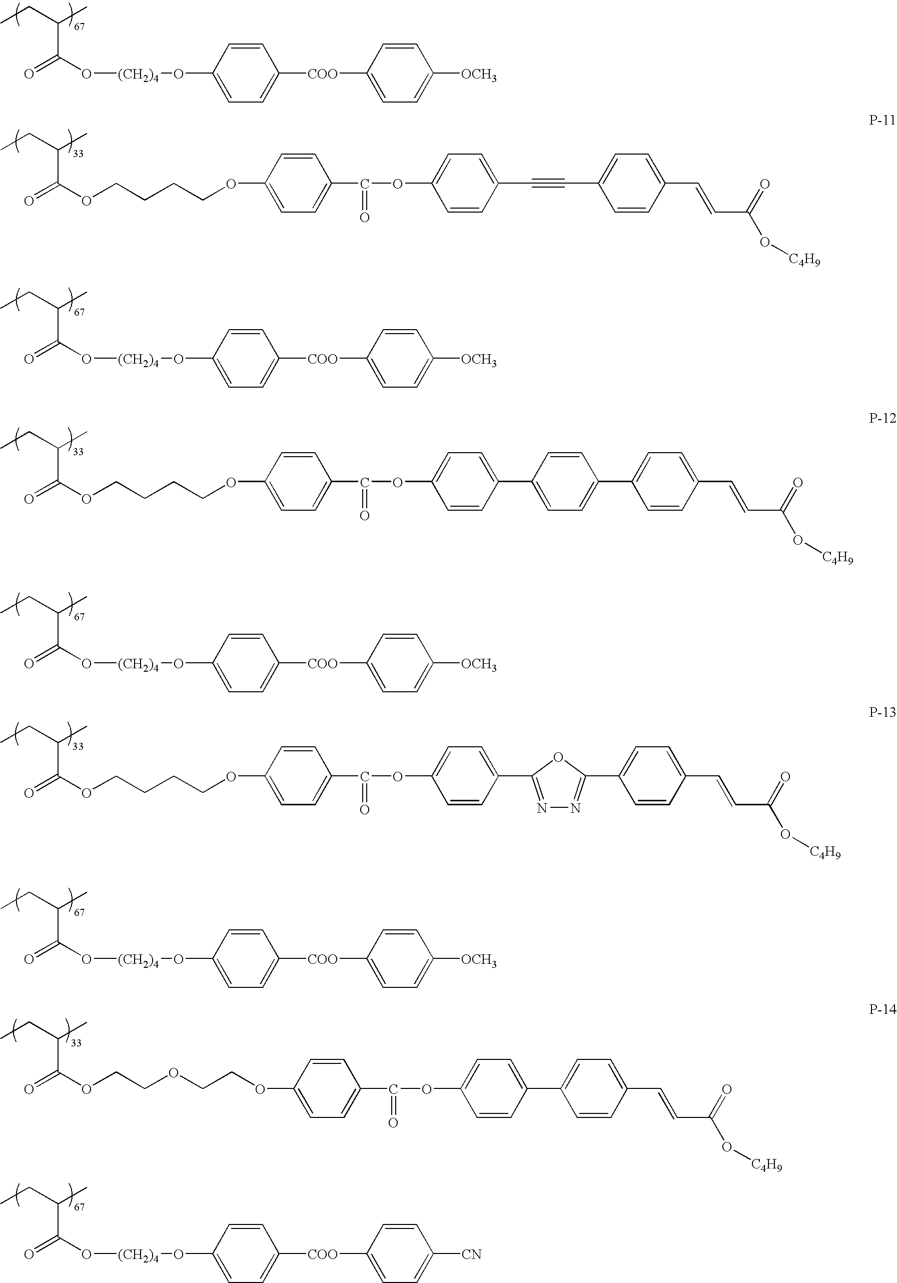

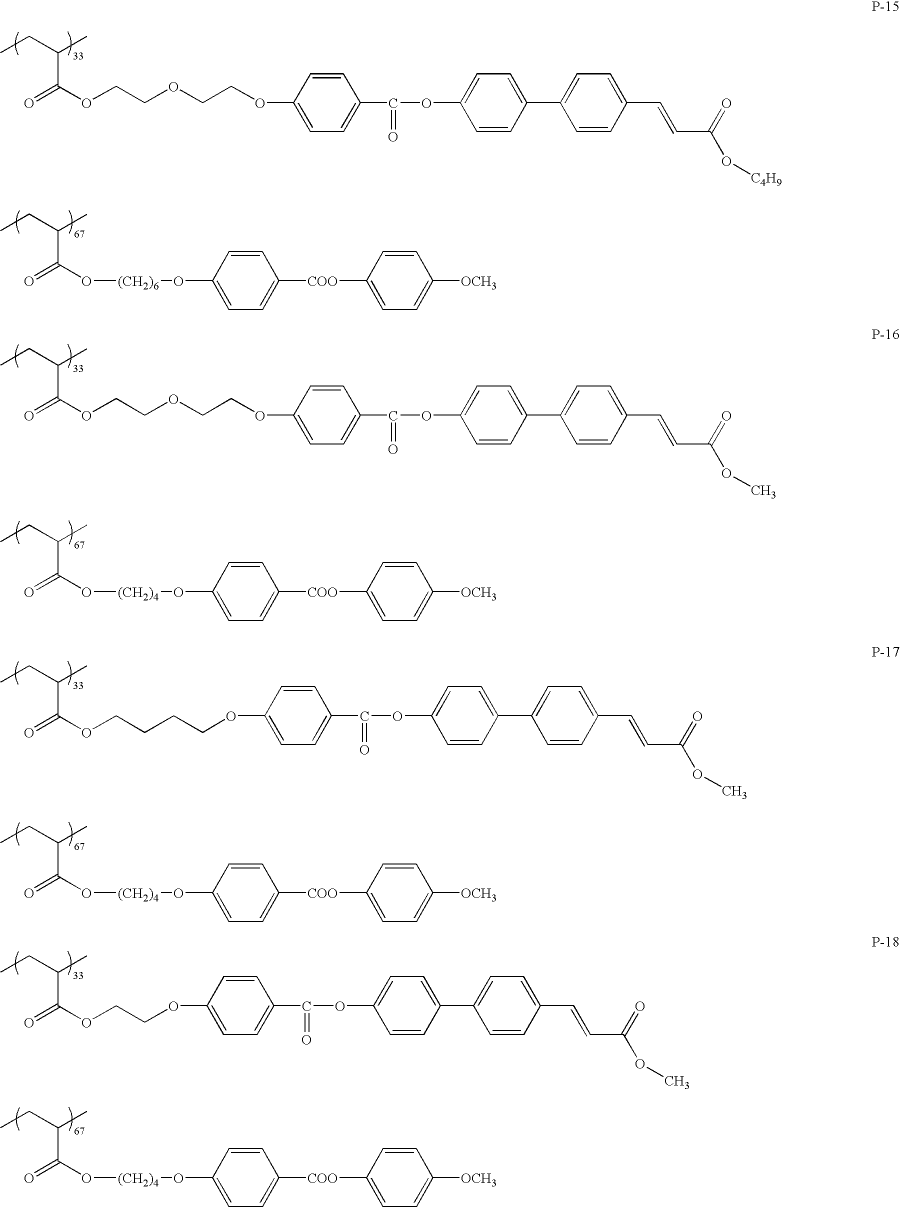

The polymer having the partial structure represented by formula (1), for example, the polymers having the repeating units represented by the formulae (1)′ and (2) may be prepared according to any method. For example, polymerization methods such as cationic polymerization and radical polymerization using vinyl group, or anionic polymerization may be used, among these, radical polymerization is particularly preferable in terms of wide adaptability. Polymerization initiator usable herein for radical polymerization may be any publicly-known compound such as radical thermal polymerization initiator, radical photo-polymerization initiator and so forth, wherein the radical thermal polymerization initiator is particularly preferable. The radical thermal polymerization initiator is a compound capable of producing a radical when heated at a temperature above the decomposition temperature. Examples of such radical thermal polymerization initiator include diacyl peroxides (acetyl peroxide, benzoyl peroxide, etc.), ketone peroxides (methyl ethyl ketone peroxide, cyclohexanone peroxide, etc.); hydroperoxides (hydrogen peroxide, tert-butyl hydroperoxide, cumene hydroperoxide, etc.); dialkyl peroxides (di-tert-butyl peroxide, dicumyl peroxide, dilauroyl peroxide, etc.); peroxyesters (tert-butyl peroxyacetate, tert-butyl peroxypivalate, etc.); azo compounds (azobisisobutyronitrile, azobis(isovaleronitrile), etc.); and persulfate salts (ammonium persulfate, sodium persulfate, potassium persulfate, etc.). Only a single species of these radical thermal polymerization initiators may independently be used, or two or more species of them may be used in combination.

The methods of radical polymerization are not specifically limited, wherein emulsion polymerization, suspension polymerization, bulk polymerization, and solution polymerization may be adoptable. Solution polymerization, which is a typical method of radical polymerization, will further specifically be explained. Outlines of the other polymerization methods are equivalent, wherein details of which may be found in “Kobunshi Kagaku Jikken Ho (Methods in Polymer Chemistry)”, edited by the Society of Polymer Science, Japan (published by Tokyo Kagaku Dozin Co., Ltd., 1981) and so forth.

The solution polymerization may be carried out in organic solvent. The organic solvent may arbitrarily be selected, so far as the purposes and effects of the present invention will not be impaired. The organic solvent is preferably an organic compound having a boiling point under the atmospheric pressure of 50 to 200° C., and is preferably such as capable of homogeneously dissolving the individual constituents. Preferable examples of the organic solvents include alcohols such as isopropanol and butanol; ethers such as dibutyl ether, ethylene glycol dimethylether, tetrahydrofuran, and dioxane; ketones such as acetone, methyl ethyl ketone, methyl isobutyl ketone, and cyclohexanone; esters such as ethyl acetate, butyl acetate, amyl acetate, and γ-butyrolactone; aromatic hydrocarbons such as benzene, toluene, and xylene; dimethylacetamide; dimethylformamide; and N-methylpyrrolidone. Only a single species of, or two or more species of these organic solvents may be used in an independent manner or in a combined manner. In the terms of solubility of the monomers and produced polymers, also water-mixed organic solvent, based on combined use of the organic solvent with water, is usable.

Although conditions for solution polymerization are not specifically limited, heating is preferably carried out within a temperature range of 50 to 200° C. for 10 minutes to 30 hours. In order to prevent the produced radical from being deactivated, the atmosphere is preferably purged with an inert gas not only, of course, in the process of solution polymerization, but also before the start of solution polymerization. The gas adoptable herein for purging is generally nitrogen gas.

Radical polymerization with the aid of a chain transfer agent may be preferable, in view of obtaining the polymer within the range of preferable molecular weight. Examples of the chain transfer agent adoptable herein include mercaptans (for example, octylmercaptan, decylmercaptan, dodecylmercaptan, tert-dodecylmercaptan, octadecyl mercaptan, thiophenol, p-nonyl thiophenol, etc.), polyhalogenated alkyls (for example, carbon tetrachloride, chloroform, 1,1,1-trichloroethane, 1,1,1-tribromooctane, etc.), and low-activity monomers (α-methylstyrene, α-methylstyrene dimer, etc.), wherein mercaptans having 4 to 16 carbon atoms are preferable. Amount of use of these chain transfer agents is considerably affected by activity of the chain transfer agent, combinations with the monomers, polymerization conditions and so forth, and needs fine control, wherein general amount may be adjusted to 0.01 mol % to 50 mol % or around, more preferably 0.05 mol % to 30 mol %, and especially preferably 0.08 mol % to 25 mol %, relative to the total mole number of monomers to be used. The chain transfer agent may reside in the system in the process of polymerization together with the monomers for which the degree of polymerization should be controlled, without limitation on the method of addition thereof. The agent may be added as being dissolved in the monomer, or may be added independently from the monomer.

Although molecular weight of the polymer is not specifically limited, not only those having a molecular weight of 10,000 or larger, with which the products may generally be understood as polymer, but also those having a molecular weight of 1,000 or larger and smaller than 10,000, with which the products may generally be understood as quasi-polymer, and also those having a degree of polymerization of 2 to 20 or around, with which the products may generally be understood as oligomer, may be included [“Iwanami Rikagaku Jiten (Iwanami Science Dictionary)”, enlarged 3rd edition, edited by Bunichi Tamamushi et al., p. 449, published by Iwanami Shoten, Publishers, 1982]. In other words, “high-molecular-weight substance” and “polymer” in the description mean those having a molecular weight of 1,000 or larger, and having a degree of polymerization of 20 or larger. The polymer preferably has a weight-average molecular weight of 1,000 to 1,000,000, more preferably 1,000 to 500,000, and still more preferably 5,000 to 100,000. The weight-average molecular weight may be measured by gel permeation chromatography (GPC) as a value relative to polystyrene (PS) standard value.

In particular, increase in the molecular weight of the polymer having the partial structure represented by formula (1) successfully increases the Nz value described later. Polymer usable in formation of the optically anisotropic film having an Nz value of 1.1 to 7.0 preferably has a weight-average molecular weight of 20,000 to 250,000.

The optically anisotropic film of the present invention may be composed solely of the compound having the partial structure represented by formula (1), or may contain any material other than the compound having the partial structure represented by formula (1) to as much as the effects of the present invention will not be impaired. In the optically anisotropic film, content of the compound having the partial structure represented by formula (1) is preferably 50 to 100% by mass, and more preferably 80 to 100% by mass.

The optically anisotropic film may contain at least one species of liquid crystalline compound.

In general, liquid crystalline compound may be classified into those of rod-like type and discotic type based on the geometry thereof. Each type has low-molecular type and polymer type. The polymer type generally means those having degrees of polymerization of 100 or larger [“Kobunshi Butsuri/So-ten'i Dinamikusu (Polymer Physics/Phase Transition Dynamics)”, Masao Doi, p. 2, published by Iwanami Shoten, Publishers, 1992]. In this embodiment, both of the liquid crystalline compounds may be adoptable, wherein the rod-like liquid crystalline compound may more preferably be used. Two or more species of the rod-like liquid crystalline compound may be used. In terms of successfully reducing temperature- and moisture-dependent changes, the rod-like liquid crystalline compound having polymerizable group(s) may be more preferable. For the embodiments wherein two or more species of the liquid crystalline compound are used, at least one of which preferably has two or more polymerizable groups in one molecule.

Examples of the rod-like liquid-crystalline compound include azomethine compounds, azoxy compounds, cyanobiphenyl compounds, cyanophenyl esters, benzoate esters, cyclohexanecarboxylic acid phenyl esters, cyanophenylcyclohexane compounds, cyano-substituted phenylpyrimidine compounds, alkoxy-substituted phenylpyrimidine compounds, phenyldioxane compounds, tolane compounds and alkenylcyclohexylbenzonitrile compounds. Not only the low-molecular-weight, liquid-crystalline compound as listed in the above, high-molecular-weight, liquid-crystalline compound may also be used. High-molecular-weight liquid-crystalline compounds may be obtained by polymerizing low-molecular-weight liquid-crystalline compounds having at least one polymerizable group. Among such low-molecular-weight liquid-crystalline compounds, liquid-crystalline compounds represented by formula (I) are preferred.

Q¹-L¹-A¹-L³-M-L⁴-A²-L²-Q² Formula (I)

In the formula, Q¹ and Q² respectively represent a reactive group. L¹, L², L³ and L⁴ respectively represent a single bond or a divalent linking group, and it is preferred that at least one of L³ and L⁴ represents —O—CO—O—. A¹ and A² respectively represent a C₂₋₂₀ spacer group. M represents a mesogen group.

The rod-like liquid crystal compound, having a reactive group, represented by formula (I), will be further described in detail hereinafter. In formula (I), Q¹ and Q² respectively represent a polymerizable group. The polymerizable groups capable of addition polymerization or condensation polymerization are preferable. Using the compound having a polymerizable group (more specifically Z) in the partial structure represented by formula (1), the polymerizable group in the rod-like liquid crystal compound may be selected from the polymerizable groups capable of polymerizing with the polymerizable group, Z, in formula (1).

Examples of polymerizable groups are shown below.

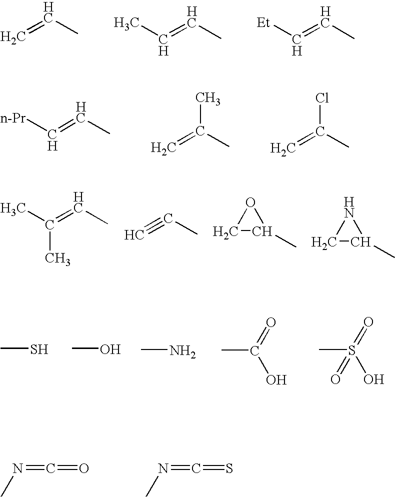

L¹, L², L³ and L⁴ independently represent a divalent linking group, and preferably represent a divalent linking group selected from the group consisting of —O—, —S—, —CO—, —NR²—, —CO—O—, —O—CO—O—, —CO—NR²—, —NR²—CO—, —O—CO—, —O—CO—NR²—, —NR²—CO—O— and —NR²—CO—NR²—. R² represents a C₁₋₇ alkyl group or a hydrogen atom. It is preferred that at least one of L³ and L⁴ represents —O— or —O—CO—O— (carbonate group). It is preferred that Q¹-L¹ and Q²-L²- are respectively CH₂═CH—CO—O—, CH₂═C(CH₃)—CO—O— or CH₂═C(Cl)—CO—O—CO—O—; and it is most preferred they are respectively CH₂═CH—CO—O—.

In the formula, A¹ and A² preferably represent a C₂₋₂₀ spacer group. It is more preferred that they respectively represent C₂₋₁₂ aliphatic group, and much more preferred that they respectively represent a C₂₋₁₂ alkylene group. The spacer group is preferably selected from chain groups and may contain at least one unadjacent oxygen or sulfur atom. And the spacer group may have at least one substituent such as a halogen atom (fluorine, chlorine or bromine atom), cyano, methyl and ethyl.

Examples of the mesogen represented by M include any known mesogen groups. The mesogen groups represented by a formula (II) are preferred.

—(—W¹-L⁵)ₙ-W²— Formula (II)

In the formula, W¹ and W² respectively represent a divalent cyclic aliphatic group, a divalent aromatic group or a divalent hetero-cyclic group; and L⁵ represents a single bond or a linking group. Examples of the linking group represented by L⁵ include those exemplified as examples of L¹ to L⁴ in the formula (I) and —CH₂—O— and —O—CH₂—. In the formula, n is 1, 2 or 3.

Examples of W¹ and W² include 1,4-cyclohexanediyl, 1,4-phenylene, pyrimidine-2,5-diyl, pyridine-2,5-diyl, 1,3,4-thiazole-2,5-diyl, 1,3,4-oxadiazole-2,5-diyl, naphtalene-2,6-diyl, naphtalene-1,5-diyl, thiophen-2,5-diyl, pyridazine-3,6-diyl. 1,4-cyclohexanediyl has two stereoisomers, cis-trans isomers, and the trans isomer is preferred. W¹ and W² may respectively have at least one substituent. Examples the substituent include a halogen atom such as a fluorine, chlorine, bromine or iodine atom; cyano; a C₁₋₁₀ alkyl group such as methyl, ethyl and propyl; a C₁₋₁₀ alkoxy group such as methoxy and ethoxy; a C₁₋₁₀ acyl group such as formyl and acetyl; a C₂₋₁₀ alkoxycarbonyl group such as methoxy carbonyl and ethoxy carbonyl; a C₂₋₁₀ acyloxy group such as acetyloxy and propionyloxy; nitro, trifluoromethyl and difluoromethyl.

Preferred examples of the basic skeleton of the mesogen group represented by the formula (II) include, but are not to be limited to, these described below. And the examples may have at least one substituent selected from the above.

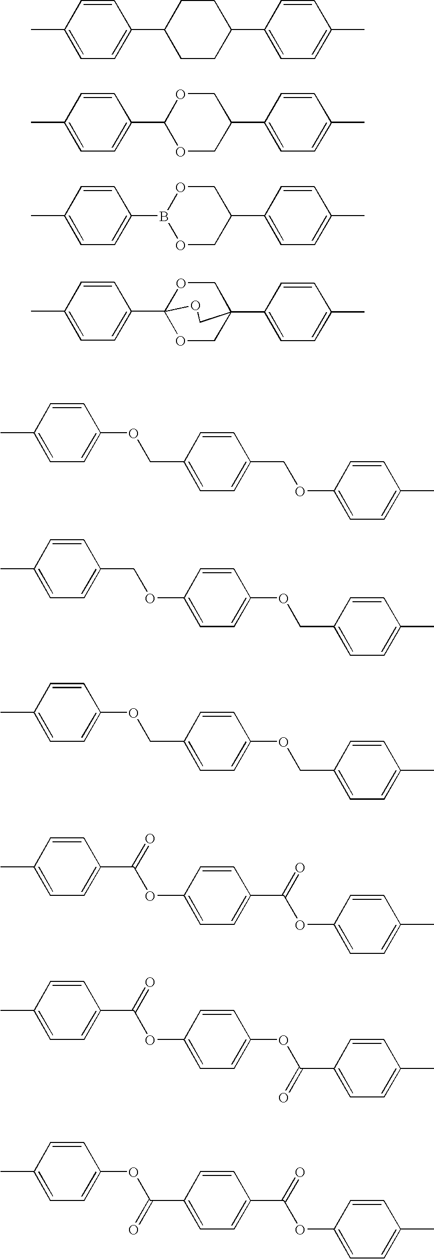

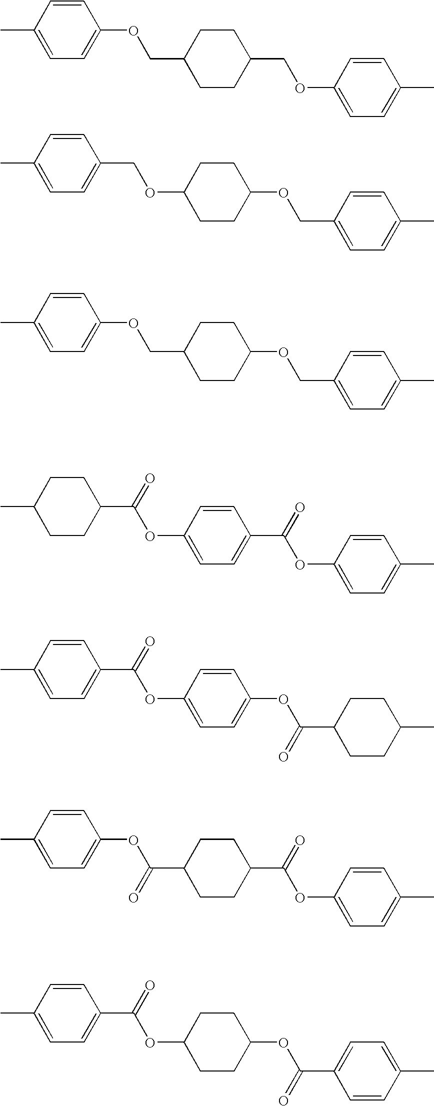

Examples the compound represented by the formula (I) include, but are not to be limited to, those described below. The compounds represented by the formula (I) may be prepared according to a method described in Japanese translation of PCT International Application No. Hei 11-513019.

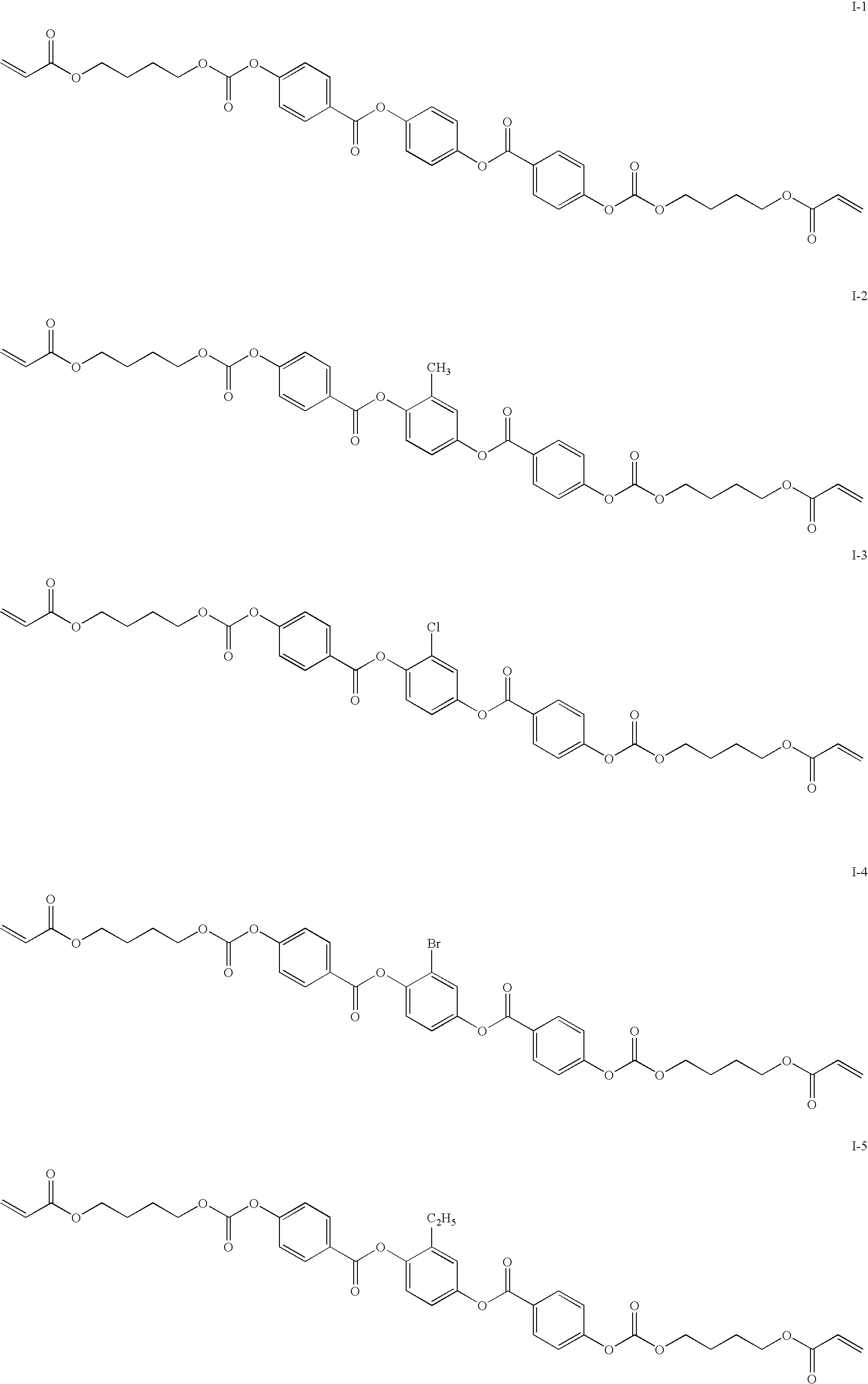

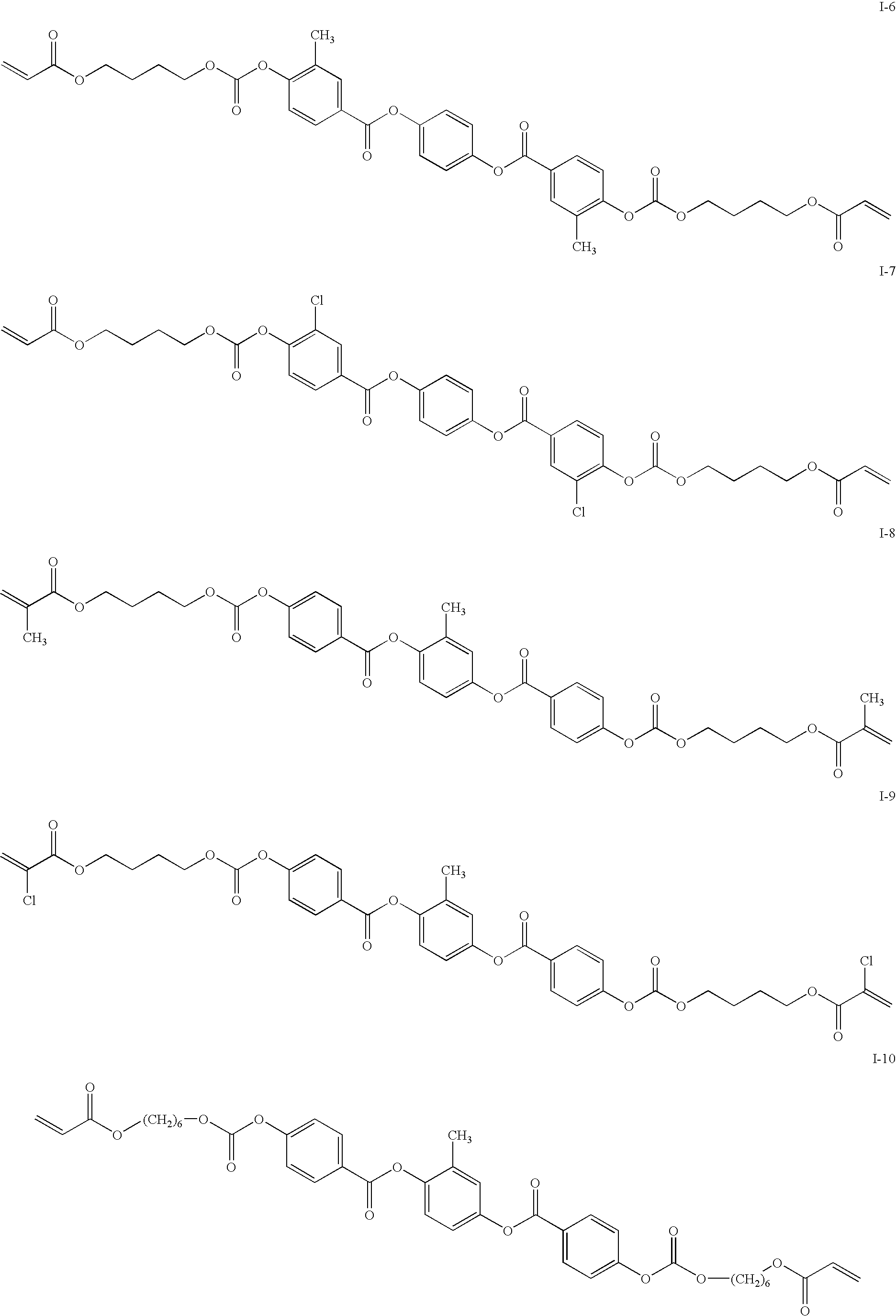

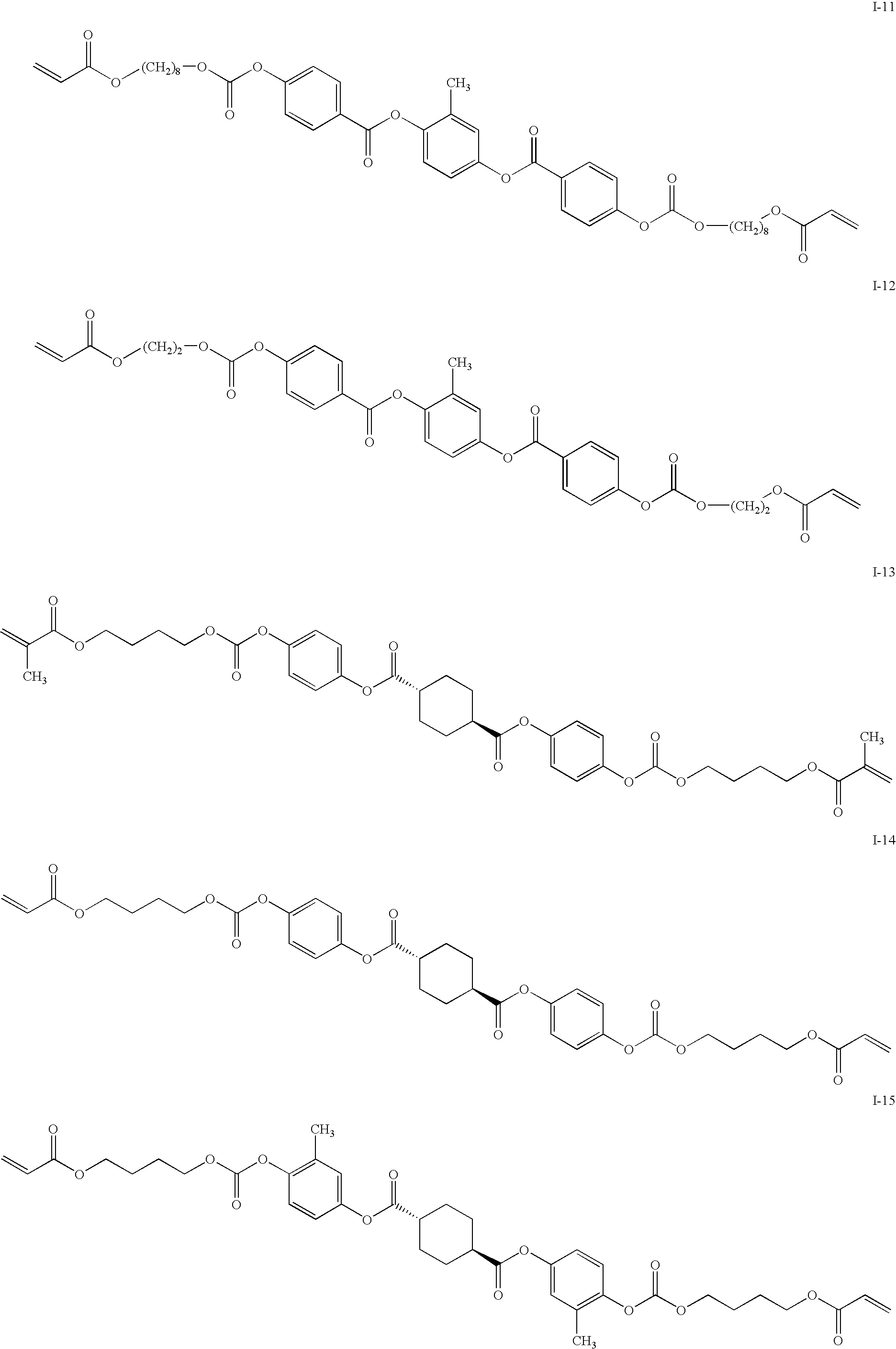

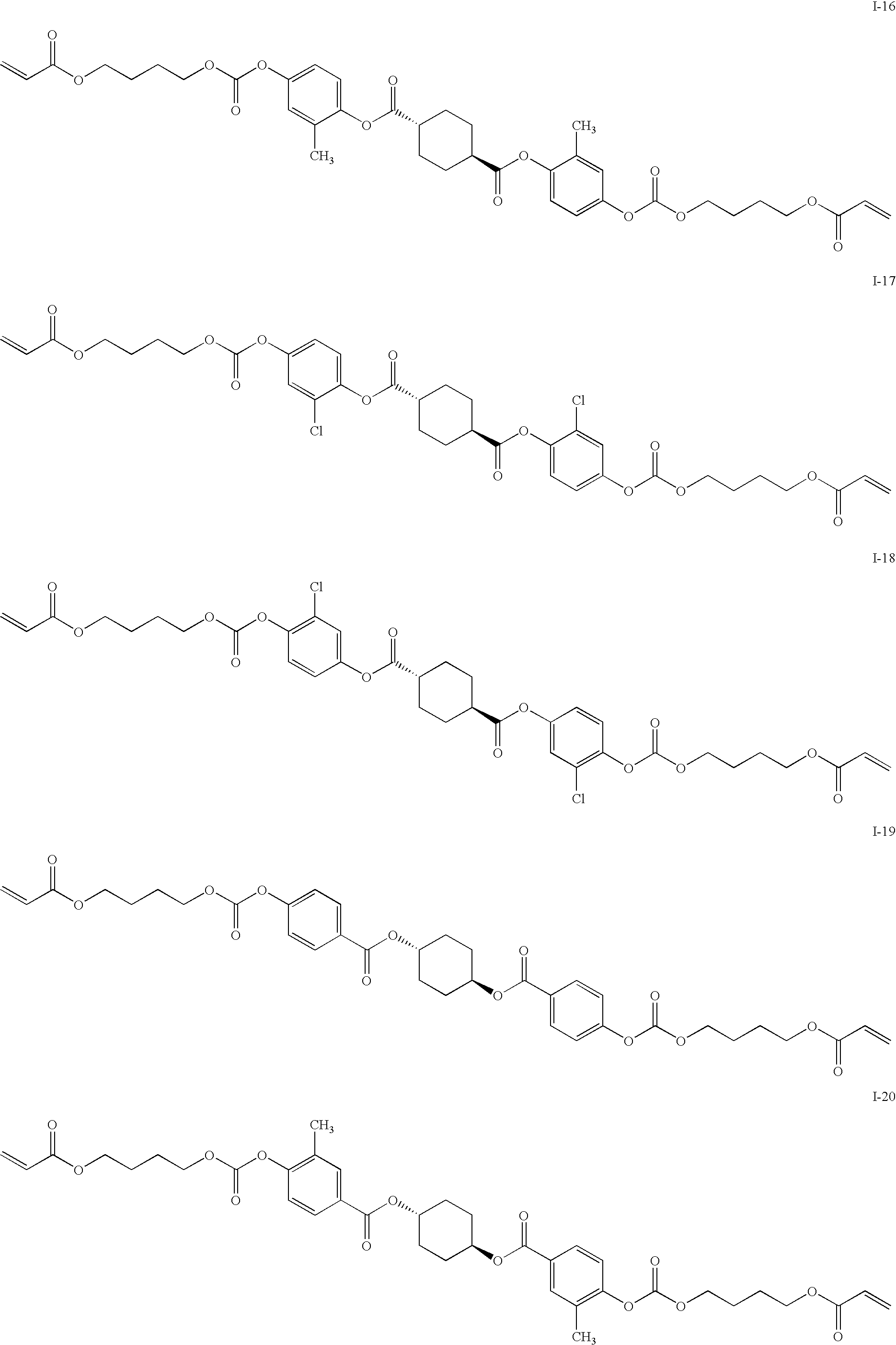

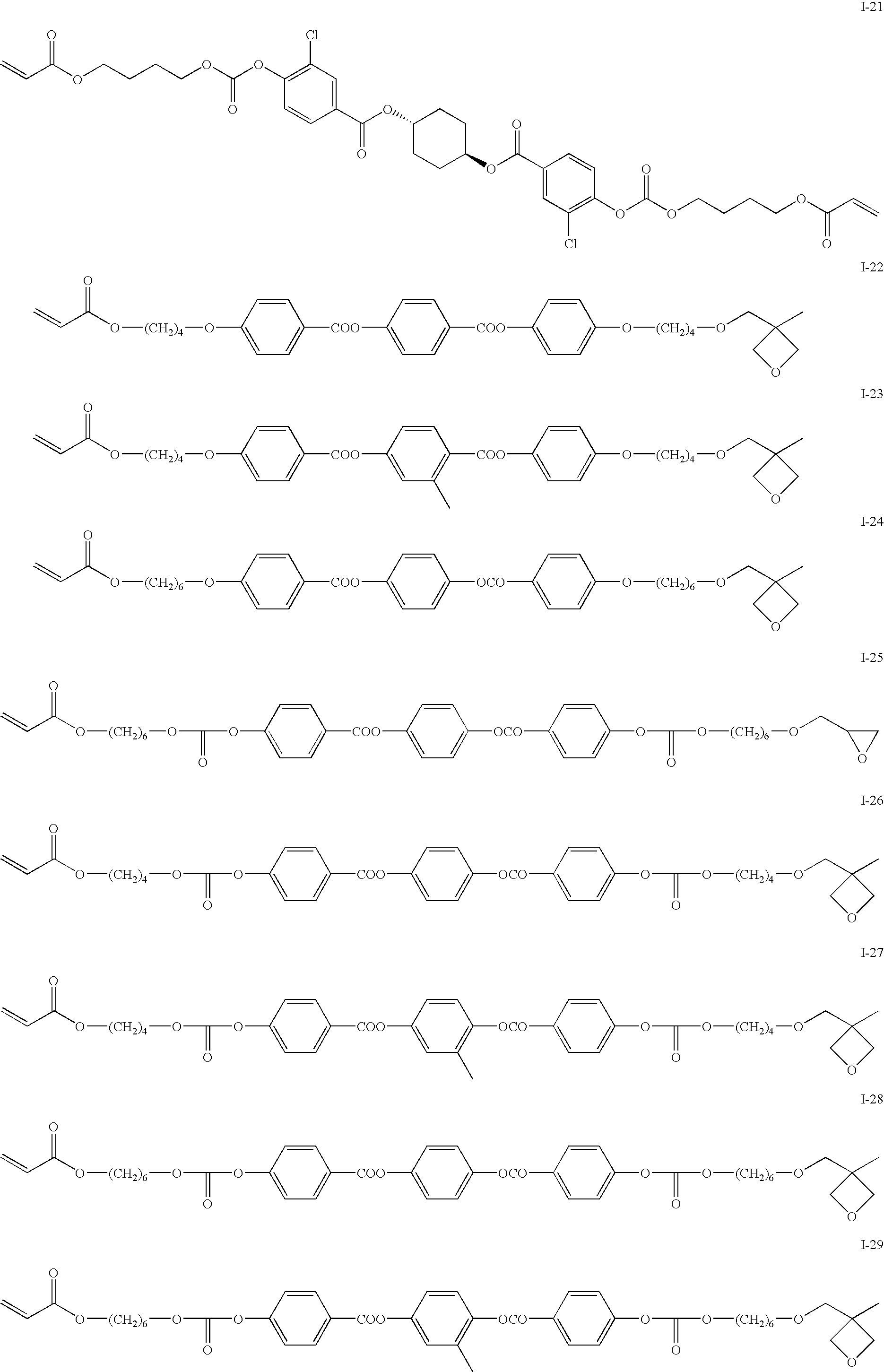

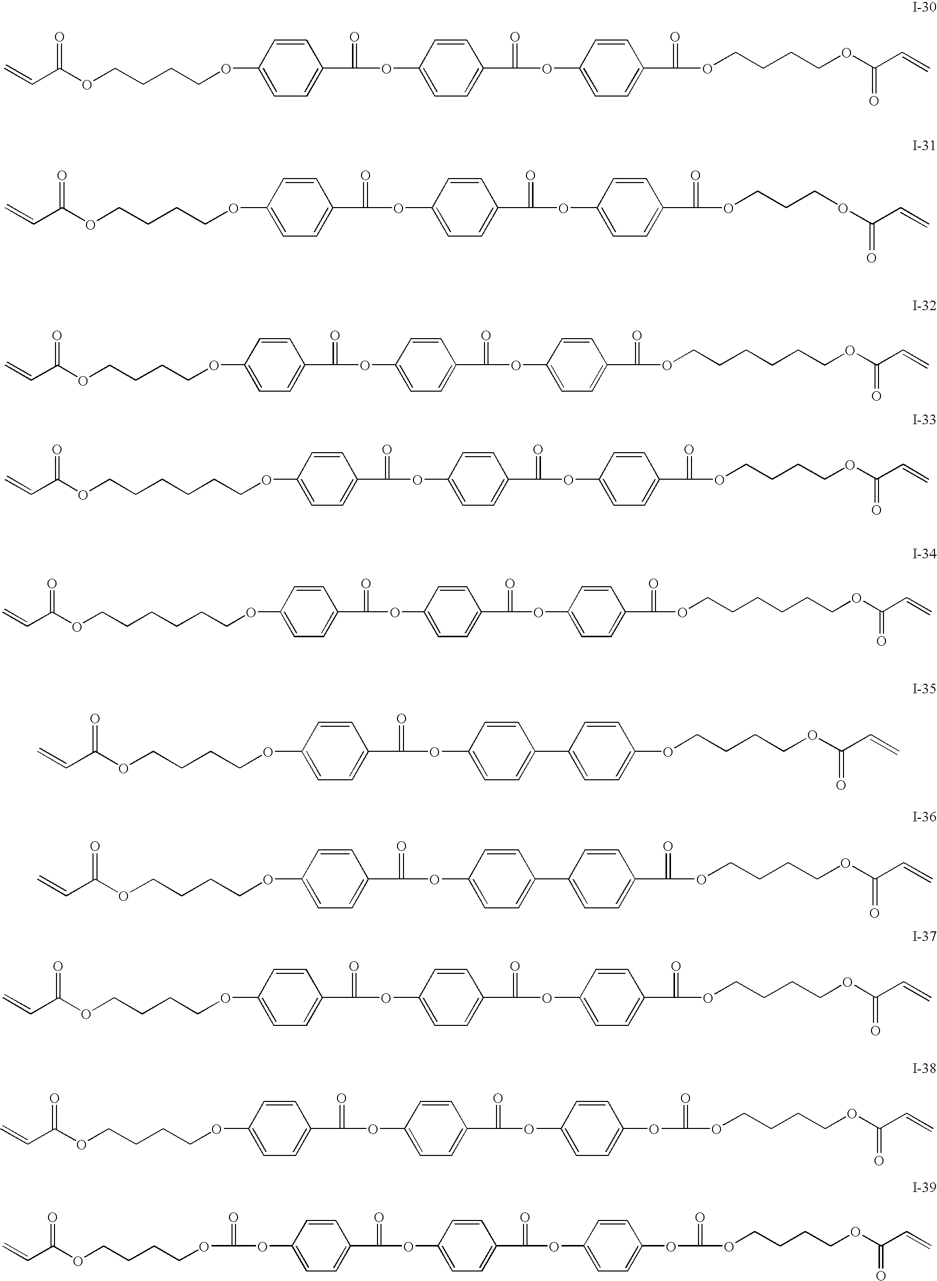

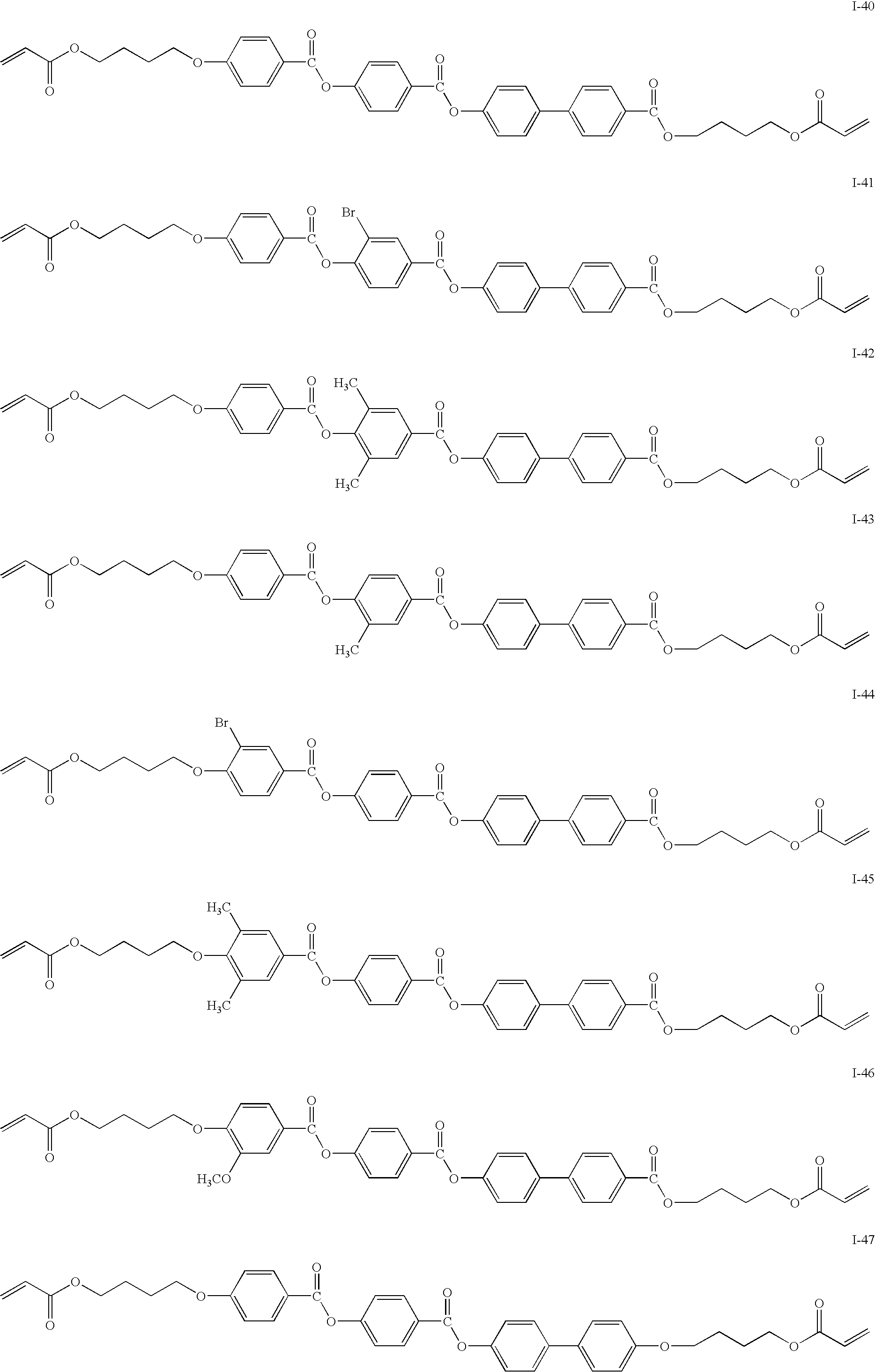

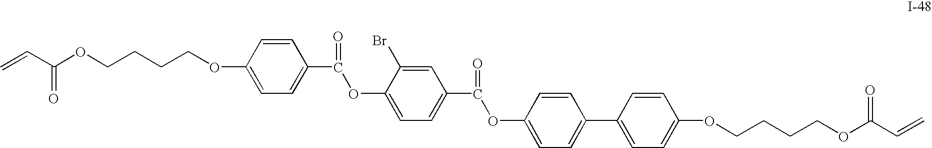

Amount of addition of the rod-like liquid crystalline compound in the optically anisotropic film is preferably 1% by mass or more, more preferably approximately 5% by mass or more, and particularly preferably approximately 10% by mass or more.

For improving the alignment ability of compound having a partial structure represented by formula (1) (or, if necessary, for improving the alignment ability of liquid crystal compound), an alignment-aid may be added to the optically anisotropic film. Examples of the alignment-aid capable of promoting a horizontal alignment of the compound having a partial structure represented by formula (1) include the compounds represented by formulas (11) to (13). The formulas will be described in detail below.

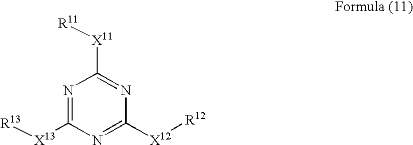

In the formula, R¹¹, R¹² and R¹³ respectively represent a hydrogen atom or a substituent; and X¹¹, X¹² and X¹³ respectively represent a single bond or a divalent linking group. Preferred examples of the substituent represented by R¹¹, R¹² or R¹³ include substituted or non-substituted alkyls (preferably non-substituted alkyls or fluoro-substituted alkyls), substituted or non-substituted aryls (preferably aryls having at least one non-substituted alkyl or fluoro-substituted alkyl), substituted or non-substituted aminos, substitute or non-substituted alkoxys, substituted or non-substituted alkylthios and halogens. X¹¹, X¹² and X¹³ respectively represent a divalent linking group; preferably represent a divalent group selected from the group consisting of an alkylene, an alkenylene, a divalent aromatic group, a divalent cyclic group, —CO—, —NR^{a}— (R^{a} represents a C₁₋₅ alkyl or a hydrogen atom), —O—, —S—, —SO—, —SO₂— and combinations thereof; and more preferably represent a divalent linking group selected from the group consisting of an alkylene, phenylene, —CO—, —NR^{a}—, —O—, —S— and —SO₂— and any combinations thereof. The number of carbon atoms of the alkylene preferably ranges from 1 to 12. The number of carbon atoms of the alkenylene preferably ranges from 2 to 12. The number of carbon atoms of the divalent aromatic group preferably ranges from 6 to 10.

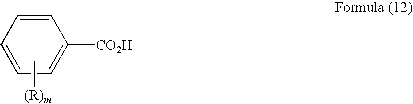

In the formula, R represents a substituent, m is an integer from 0 to 5. When m is 2 or more, plural R may be same or different each other. Preferred examples of the substituent represented by R are same as those exemplified as examples of R¹¹, R¹² or R¹³. In formula (12), m preferably represents an integer ranging from 1 to 3, and is more preferably 2 or 3.

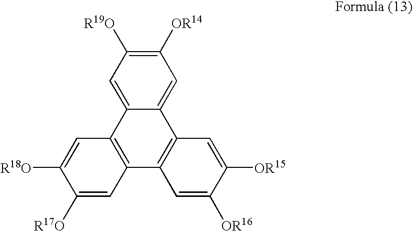

In the formula, R¹⁴, R¹⁵, R¹⁶, R¹⁷, R¹⁸ and R¹⁹ respectively represent a hydrogen atom or a substituent. Preferred examples of the substituent represented by R¹⁴, R¹⁵, R¹⁶, R¹⁷, R¹⁸ and R¹⁹ are same as those exemplified as examples of R¹¹, R¹² or R¹³ in formula (11). Examples of the horizontal alignment agent, which can be used in the present invention, include those described in JPA No. 2005-099248, paragraphs [0092]-[0096], and the methods for preparing such compounds are described in the document.

The amount of the compound represented by the formula (11), (12) or (13) is preferably from 0.01 to 20 mass %, more preferably from 0.01 to 10 mass % and much more preferably from 0.02 to 1 mass % with respect to the total mass of the compound having a partial structure represented by formula (1). One type compound may be selected from formula (11), (12) or (13) and used singly, or two or more type of compounds may be selected from formula (11), (12) or (13) and used in combination.

One example of a method of preparing the optically anisotropic film of the present invention is as follows. A composition containing the compound having the partial structure represented by formula (1) is prepared, applied to a surface, dried, and then irradiated with polarized light, so that the partial structure represented by formula (1) is aligned and expresses birefringence. Being irradiated with polarized light, the partial structure represented by formula (1) is aligned, and retardation in plane develops. The irradiation of polarized light is preferably carried out after the composition containing the compound was applied to a surface and then dried, and before any other treatment (curing, for example). Energy of irradiation in the irradiation of polarized light may preferably be 20 mJ/cm² to 10 J/cm², and more preferably 100 to 800 mJ/cm².

In this step, the illumination intensity is preferably from 20 to 1000 mW/cm², more preferably from 50 to 500 mW/cm², and much more preferably from 100 to 350 mW/cm². Light to be used in this step preferably has a peak within the range from 300 to 450 nm, and more preferably within the range from 350 to 400 nm.

The layer of the composition may be heated after being irradiated with polarized light. In such a case, the alignment may be matured, and therefore larger retardation in plane may be obtained.

The temperature of the heating step is preferably from 50° C. to 250° C., more preferably from 50° C. to 200° C., much more preferably from 70° C. to 170° C.

The period for heating is not limited to any range. According to one example, in which an optically anisotropic film having Nz value ranging from 1.1 to 7 is prepared, the period for heating is preferably from one second to five minutes, but is not limited to the range.

After birefringence emerging, preferably after being heated, the layer of the composition may be irradiated with polarized or non-polarized light. This step may be carried out for promoting the reaction of the polymerizable groups of any ingredient in the layer and for further hardening the layer, and therefore may contribute to improving the thermal durability. In this step, the irradiation energy is preferably from 20 mJ/cm² to 10 J/cm², further preferably from 100 to 800 mJ/cm². The illumination intensity is preferably from 20 to 1000 mW/cm², more preferably from 50 to 500 mW/cm², further preferably from 100 to 350 mW/cm². In the embodiments of irradiating with polarized light, light having a peak at a wavelength from 300 to 450 nm is preferable, and light having a pea at a wavelength from 350 to 400 nm is more preferable. In the embodiments of irradiating with non-polarized light, light having a peak at a wavelength from 200 to 450 nm is preferable, and light having a pea at a wavelength from 250 to 400 nm is more preferable.

As has been described in the above, using any of the curable compounds and the like having polymerizable group(s), the polymerization of the composition may progress after birefringence is generated. The polymerization reaction usable herein may be either of thermal polymerization reaction making use of thermal polymerization initiator, and photo polymerization reaction making use of photo-polymerization initiator, wherein photo-polymerization reaction is more preferable. For smooth proceeding of the polymerization reaction, the composition preferably contains the polymerization initiator. Examples of the thermal polymerization initiator to be used in radical polymerizations include azobisisobutyronitrile. Examples of the photo-polymerization initiator to be used in radical polymerizations include α-carbonyl compounds (those described in U.S. Pat. Nos. 2,367,661 and 2,367,670), acyloin ethers (those described in U.S. Pat. No. 2,448,828), α-hydrocarbon-substituted aromatic acyloin compounds (those described in U.S. Pat. No. 2,722,512), polynuclear quinone compounds (those described in U.S. Pat. Nos. 3,046,127 and 2,951,758), combinations of triarylimidazole dimer and p-aminophenyl ketone (those described in U.S. Pat. No. 3,549,367), acrydine and phenazine compounds (those described in JPA No. S60-105667 and U.S. Pat. No. 4,239,850), and oxadiazole compounds (those described in U.S. Pat. No. 4,212,970).

The amount of the photo-polymerization initiator is preferably 0.01 to 20% by mass, and more preferably 0.5 to 5% by mass, with respect to the total mass of the solid content of the composition. The irradiation energy is preferably from 20 mJ/cm² to 10 J/cm², and further preferably from 100 to 800 mJ/cm². for promoting the photo-polymerization, irradiation of light may be carried out under a nitrogen atmosphere or heat.

The composition to be used for preparing the optically anisotropic film of the invention is preferably prepared as a coating liquid. Examples of the solvent to be used for preparing the coating liquid include organic solvents such as amides (e.g., N,N-dimethylformamide), sulfoxides (e.g., dimethylsulfoxide), heterocyclic compounds (e.g., pyridine), hydrocarbons (e.g., benzene, hexane), alkyl halides (e.g., chloroform, dichloromethane), esters (e.g., methyl acetate, butyl acetate), ketones (e.g., acetone, methyl ethyl ketone), and ethers (e.g., tetrahydrofuran, 1,2-dimethoxyethane). Alkyl halides and ketones are preferred. Two or more types of organic solvent may be used in a mixture.

The composition may be applied to a surface according to any method. As described later, the optically anisotropic film of the present invention may be disposed in a liquid crystal cell, as being formed in the regions corresponded to the individual pixels, and in such a case, the composition is preferably applied to a surface according to any inkjet system.

The composition may also be applied to a rubbed surface. According to this method, molecules are aligned in a predetermined direction along the rubbing direction, and then irradiated with polarized light, so that the desired optical characteristics may be obtained. In this method, the polarized light may preferably be irradiated at an angle different from the rubbing direction.

Thickness of the optically anisotropic film is preferably 0.1 to 20 μm, and more preferably 0.5 to 10 μm.

### [Optical Characteristics and Applications of Optically Anisotropic Film]

The optically anisotropic film of the present invention exhibits retardation in plane Re, and it possibly satisfies the characteristics required for a monoaxial film such as A-plate, or biaxial film.

Although an A-plate is generally understood as satisfying an optical characteristic given by nx>ny=nz, it will be understood in the description that also those characterized by a Re(550) of approximately 20 to 300 nm, and an Nz value (where, Nz=Rth(550)/Re(550)+0.5) of approximately 0.9 to 1.1 may be included in the category of an A-plate. The optically anisotropic film of the present invention may function as an A-plate, so that it may be usable in optical compensation of liquid crystal display devices, in place of an A-plate which has conventionally been used, and is particularly suitable for optical compensation of VA-mode liquid crystal display devices. In the embodiment wherein the optically anisotropic film of the present invention is used as the A-plate (for example, used for optical compensation of VA-mode liquid crystal display devices), Re(550) preferably falls within the range from 50 to 200 nm, and more preferably the range from 70 to 200 nm.

A biaxial film is generally understood as having different values for all of nx, ny and nz. One example of the film may have an optically characteristic given by nx>ny>nz. The optically anisotropic film of the present invention may function as a biaxial film having a Re(550) of approximately 20 to 300 nm, and an Nz value (where, Nz=Rth(550)/Re(550)+0.5) of approximately 1.1 to 7.0. In other words, the optically anisotropic film of the present invention may be usable in optical compensation of the liquid crystal display devices, as a substitute for the biaxial film which has conventionally been used, and may be particularly suitable for optical compensation of VA-mode liquid crystal display devices. In the embodiment wherein the optically anisotropic film of the present invention is used as a biaxial film (typically for optical compensation of VA-mode liquid crystal display devices), the Nz value of the film is preferably be 1.5 to 7.0, and more preferably 2.0 to 6.0. Re(550) is preferably 20 to 300 nm, more preferably 20 to 200 nm, and still more preferably 20 to 100 nm.

Another example of the biaxial film is a film having an optical characteristic given by nx>nz>ny. The optically anisotropic film of the present invention may function also as the biaxial satisfying the above-described relation, while being adjusted in the ratio of polymerization and molecular weight of the polymer used for the fabrication. In other words, the optically anisotropic film of the present invention in this embodiment may be usable in optical compensation of the liquid crystal display devices as a substitute of the biaxial film, which satisfies nx>nz>ny and has conventionally been used, and may be particularly suitable for optical compensation of the IPS-mode liquid crystal display devices. The optically anisotropic film adoptable to the IPS-mode liquid crystal display devices preferably has an Nz value of 0.1 to 0.9, and more preferably 0.3 to 0.7, and Re(550) of 200 to 400 nm.

The optically anisotropic film of the present invention can exhibits desired optical characteristics by irradiation with polarized light, without needing any alignment film, so that it may advantageously be formed in every micro region, in particular in the regions corresponded to the individual pixels in the liquid crystal cell. Of course, also for the embodiment wherein the optically anisotropic film of the present invention is formed in the regions corresponded to the individual pixels in the liquid crystal cell, the alignment film may be formed in the individual regions, the liquid crystal molecules are once aligned on the alignment film, and then subjected to irradiation of polarized light.

In the embodiment where the optically anisotropic film is formed in the liquid crystal cell, optical characteristics of the optically anisotropic film may preferably be adjusted to achieve optimum optical characteristics for optical compensation upon incidence of R light, G light and B light. More specifically, the optically anisotropic film to be formed in the region corresponded to the R layer of the color filter may preferably be optimized in the optical characteristics thereof, so as to achieve the best viewing angle compensation with respect to incident R light, the optically anisotropic film to be formed in the region corresponded to the G layer of the color filter may preferably be optimized in the optical characteristics thereof, so as to achieve the best viewing angle compensation with respect to incident G light, and the optically anisotropic film to be formed in the region corresponded to the B layer of the color filter may preferably be optimized in the optical characteristics thereof, so as to achieve the best viewing angle compensation with respect to incident B light. The optical characteristics of the optically anisotropic film may be adjustable to preferable ranges, typically by controlling any species of the compound having the partial structure represented by formula (1), species and amount of addition of an alignment control agent, thickness of the film, and conditions for irradiation of polarized light.

Alternatively, the optically anisotropic film itself may be also used as a color filter. In this embodiment, the composition for forming the optically anisotropic film may be added with pigments or the like of the individual colors of R, G and B.

One example of the method of forming the optically anisotropic film of the present invention on the liquid crystal cell substrate, in the regions thereof corresponded to the individual pixels, is a method employing any inkjet system. More specifically, the optically anisotropic film may be produced as follows. A liquid containing the compound having the partial structure represented by formula (1) is applied to regions partitioned by a black matrix according to an inkjet system, irradiated with polarized light, so that the desired optical characteristics are obtained. And, after that, if necessary, the film is matured under heating. In order to further improve the durability, the film may be irradiated with ionized radiation, so that the polymerization of the ingredients in the film may progress and the alignment state may be fixed.

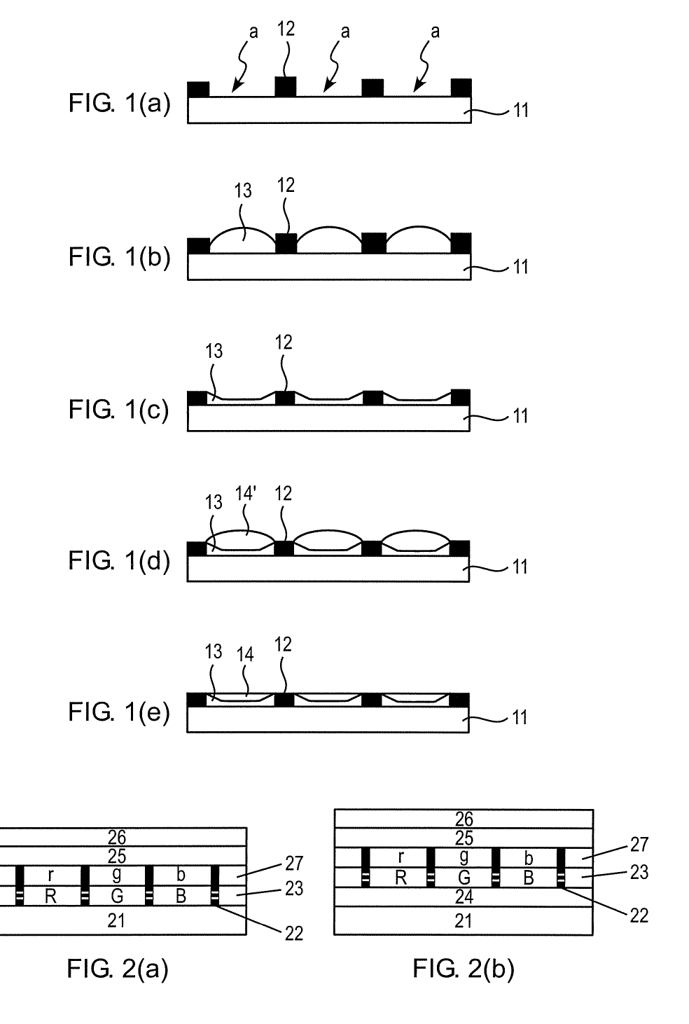

One example of the process for producing the liquid crystal cell having the optically anisotropic film of the invention therein will be described while referring to FIG. 1.

On a transparent substrate 11 composed of glass etc., a black matrix 12 (barrier walls) of dot pattern is formed using a negative type black matrix resist material according to a photo lithographic method to form plural fine areas separated by the barrier walls 12 (FIG. 1(A)). Incidentally, in the formation of the black matrix 12, there is no particular limitation on the material and process for forming the black matrix, and the black matrix may be formed according to a method other than the photo lithographic method. The pattern of black matrix 12 is not limited to the dot pattern. There is no particular limitation on the alignment of a color filter to be formed, and any of dot alignment, stripe alignment, mosaic alignment and delta alignment can be used.

The black matrix 12 is preferably subjected to plasma treatment after the formation with a gas of fluoro-containing compound (such as CF₄) so that the surface thereof is treated to be ink-rejecting. The ink-rejecting black matrix 12 may be obtained according to a method other than the above-described plasma treatment. For example, the ink-rejecting black matrix can be obtained by producing the black matrix using a material comprising an ink-rejecting agent, or using an ink-rejecting material.

Next, a fluid composition 13′ containing a compound having a partial structure represented by formula (1) is ejected by using an ink jet apparatus to the fine areas “a” separated by the black matrix 12, and the layers of the a fluid composition 13′ are formed in the fine areas “a” (FIG. 1(B)). After being ejected to the fine areas, the fluid is irradiated with polarizing light, so that birefringence is generated in the layers. In this way, optically anisotropic layers 12 are formed (FIG. 1(B)). After or before the irradiation of polarized light or during the irradiation of polarized light, the layers may be heated, and in such a case, any heating apparatus may be used.

To each optically anisotropic layer 13 formed in the manner described above as a first layer, an ink fluid 14′ is secondarily ejected (FIG. 1(D)), dried and, if desired, exposed to form a color filter layer 14 as a second layer (FIG. 1(E)).

There is no particular limitation on the ejection condition of the fluid such as ink upon forming the optically anisotropic layer 13 and color filter layer 14, but, when a fluid for forming the optically anisotropic layer and an ink for forming the color filter layer have a high viscosity, it is preferred to eject these with a reduced viscosity under room temperature or elevated temperatures (such as 20-70° C.) in terms of ejection stability. Since the variation of viscosity of the ink etc. has directly a significant influence on the droplet size and droplet ejection rate to result in an image quality degradation, the temperature of ink etc. is preferably kept as constant as possible.

An ink jet head (hereinafter, it may also be simply referred to as a head) for use in the process of the invention is not particularly limited, and publicly known various ones can be used. A head of the continuous type or dot on-demand type may be used. Among the dot on-demand type, as a thermal head, a type having such operative valve for ejection as described in JPA No. hei 9-323420 is preferred. In the case of a piezo head, for example, heads described in EP 277,703 A, EP 278,590 A etc. can be used. A head having a temperature-controlling function is preferred so that the temperature of a composition can be regulated. It is preferred that the ejection temperature is controlled so that the viscosity at ejection becomes 5-25 mPa·s, and that the composition temperature is controlled so as to give the fluctuation range of the viscosity of ±5% or less. As to the drive frequency, operation at 1-500 kHz is preferred.

The order of the optically anisotropic layer 13 and the color filter layer 14 may be interchanged, that is, the optically anisotropic layer 13 may be formed on the color filter layer 14. The embodiment can be produced by interchanging the order of the step of forming the optically anisotropic layer 13 and the step of forming the color filter layer 14 in the above example of the producing process.

The compound having a partial structure represented by formula (1) may be added to an ink composition to be used for preparing a color filter layer.

The optically anisotropic layer 13 may be formed by using a fluid, such as a solution, of the same type, or may be formed by using different fluids, such as solutions, containing materials different from each other and/or having different formulations (blending amounts) from each other so that each of them exhibits the optical anisotropy optimized relative to each hue of the color filter layer 14 that is formed thereon. When plural different fluids are used relative to hues of the color filter layer, the optically anisotropic layer 13 may be formed by carrying out the ejections of all of the fluids one after another, and then drying them concurrently, or by carrying out the set of the ejection of each fluid and drying it repeatedly. Similarly, the color filter layer 14 may be formed by carrying out the ejections of all of the ink fluids (e.g., ink fluids for preparing an R layer, G layer and B layer) one after another, and then drying them concurrently, or by carrying out the set of the ejection of each fluid and drying it repeatedly. In addition, the color of a color filter needs not to be limited to three colors of red, green and blue. A color filter may be of multi-primary colors.

Thus, the first substrate, having thereon an optically anisotropic layer 13 and a color filter layer 14 at each fine area, corresponding each pixel, separated by black matrix 12 (barrier wall), is obtained. As mentioned above, the optically layer 13 and the color filter layer 14 are formed by ejecting the fluid, which is prepared so as to exhibit a predetermined optical anisotropy, and the ink-fluid (e.g., red, green or blue ink fluid), and then drying them. After that, the first substrate is laminated with the second substrate. Before the lamination, a transparent electrode layer and/or an alignment layer may be formed on the color filter layer 14. For example, as described in JPA No. hei 11-248921 and Japanese Patent No. 3255107, it is preferred, in terms of cost reduction, to form a base by superimposing colored resin compositions forming a color filter, forming a transparent electrode thereon, and, according to need, forming a spacer by superimposing protrusions for divided alignment.

A liquid crystal material may be poured into a gap between the facing surfaces of the first and second substrates to form a liquid crystal layer; and, then, a liquid crystal cell is produced. The first substrate is preferably disposed so that the surface on which the optically anisotropic layer and the color filter layer have been formed lies inside, that is, becomes a facing surface. Then, polarizing plates, optical compensatory films etc. can be laminated on the outside surfaces of both substrates, respectively, to manufacture a liquid crystal display device.

According to the above mentioned example of the process, after forming barrier walls corresponding a black matrix, the fluid for forming an optically anisotropic layer and the ink fluids for forming a color filter layer are applied to predetermined regions by using an ink jet system; and, therefore, it is possible to form accurately the optically anisotropic layer and the color filter layer in predetermined regions on the first substrate. Consequently, the desired liquid crystal cell can be obtained, without making the construction complex, with a small number of steps.

In the description of the method of the invention, an example was adopted in which the ink ejection by an ink jet method was used to form an optically anisotropic layer and color filter layer in respective fine areas. However, the liquid crystal display device or the color filter plate of the invention is not limited to the embodiment produced by such method, and, needless to say, embodiments, in which an optically anisotropic layer and/or a color filter layer has been formed by utilizing a method other than the ink jet method, for example, a printing method or the like, also fall within the scope of the invention.

### [Liquid Crystal Cell Substrate]

The present invention relates also to a liquid crystal cell substrate having the optically anisotropic film of the invention thereon. One examples of the substrate of the invention comprises a substrate, the optically anisotropic film of the invention to be used for optical compensation of a liquid crystal cell, and a color filter layer, wherein the optically anisotropic film has the optical property optimized, in terms of viewing angle compensation of the liquid crystal cell, relative to the hue of the color filter layer (for example, for each color of R, G, B) disposed on or under thereof. There is no particular limitation on the material of the substrate provided that it is transparent; and examples include metal substrates, metal-laminated substrates, glass substrates, ceramics substrates and low-birefringent polymer films. Desirably it has a small birefringence, and glass, a low birefringence polymer or the like is used. And the substrate may have an alignment layer for controlling alignment of liquid crystal and/or a transparent electrode layer thereon.

The liquid crystal cell substrate may have a second optically anisotropic layer on its surface, which may be disposed facing outside of the cell when it is used in an LCD, opposite to its surface having the optically anisotropic film of the invention. The second optically anisotropic layer may contribute to optically compensating birefringence of a liquid crystal cell along with the optically anisotropic film of the invention disposed in the liquid crystal cell. The preferable range of the optical characteristics of the second optically anisotropic layer may be varied depending on the mode of the LCD. One example of the substrate of the invention, to be used in a VA-mode liquid crystal display device, has the optically anisotropic film of the invention functioning as an A-plate on its inner surface, and a second optically anisotropic layer functioning as a negative C-plate on its outer surface.

FIG. 2 shows a rough cross-sectional drawing of one example of a liquid crystal cell substrate of the invention.

In the substrate for the liquid crystal cell shown in FIG. 2(A), a black matrix 22 is formed on a transparent substrate 21 as the barrier wall, and a patterned color filter layer 23 and an optically anisotropic layer 27 are formed, which have been formed by ejection from an ink jet system, in fine areas separated by the barrier wall. It further has a transparent electrode layer 25 and an alignment layer 26 thereon. In FIG. 2, an embodiment is shown in which the color filter layer 23 of R, G, B has been formed, but a color filter layer composed of a layer of R, G, B, W (white), which is frequently used recently, may be formed. The optically anisotropic layer 27 is divided into respective r, g, and b regions, which have optimal retardation for respective hues of R, G, B of the filter layer 23.

Further, as shown in FIG. 2(B), a second optically anisotropic layer 24 which contribute to optical compensation along with the optically anisotropic film 27 of the invention. The second optically anisotropic layer 24 may be formed on the substrate having a color filter and the optically anisotropic film 27 of the invention thereon, or, although not shown as a drawing, it may be formed on another substrate facing the color filter substrate. On the facing substrate side, generally an electrode for driving such as a TFT array is arranged frequently, and although the layer 24 may be formed at any position on the facing substrate, in the case of active drive type having TFT, it preferably lies above a silicon layer from the viewpoint of heat resistance of the optically anisotropic layer.

### [Liquid Crystal Display Device]

The invention relates also to a liquid crystal display comprising the optically anisotropic film of the invention. The optically anisotropic film of the invention may be disposed outside of a liquid crystal cell and between a polarizing element and the liquid crystal cell, or may be disposed inside of the liquid crystal cell as described above. The liquid crystal display device of the invention may further comprise a second optically anisotropic layer contributing to optical compensation along with the optically anisotropic film of the invention.

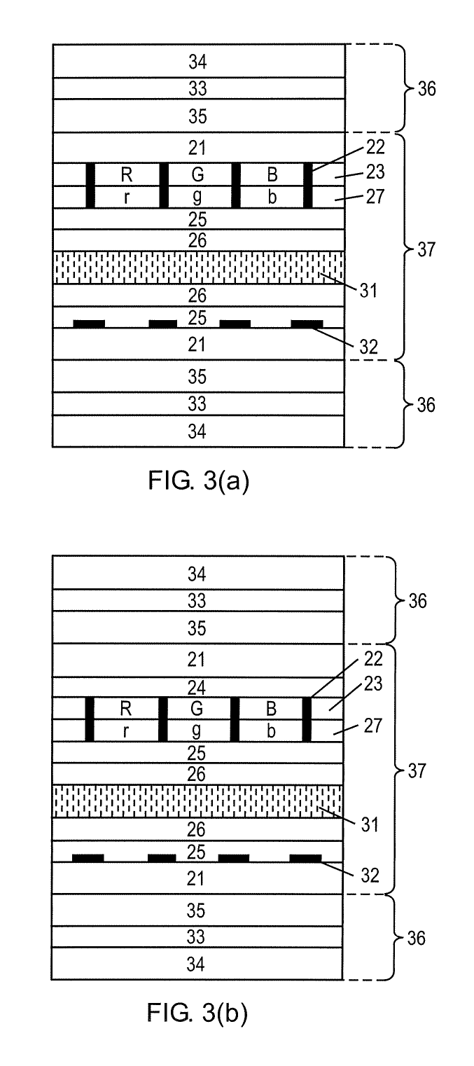

FIG. 3 is a rough cross-sectional drawing of one example of the liquid crystal display device of the invention.

Each of examples in FIGS. 3(A) and 3(B) is a liquid crystal display device having a liquid crystal cell 37 constituted by using the substrates in FIGS. 2(A) and 2(B) as the upper substrate respectively, arranging, as a facing substrate, the glass substrate 21 having a transparent electrode layer 25 with a TFT 32 and an alignment layer 26 thereon, and interposing liquid crystal 31 therebetween. On both sides of the liquid crystal cell 37, there is arranged a polarizing plate 36 composed of a polarizing layer 33 interposed between protective layers 34 and 35 composed of a cellulose acetate (TAC) film etc. The protective layer 35 on the liquid crystal cell side may be a polymer film such as a TAC film that satisfies the optical property as an optical compensatory sheet, or composed of the same polymer film as the protective layer 34. Although not shown in the drawing, in an embodiment of a reflective liquid crystal display device, only one polarizing plate is satisfactorily arranged on the viewer side, and a reflective film is provided on the backside of the liquid crystal cell or on the inside face of the downside substrate of the liquid crystal cell. Of course, a front light may be provided on the viewer side of the liquid crystal cell. Further, a semi-transparent type, in which a transmissive portion and a reflective portion are provided in one pixel of a display device, is also possible. There is no particular limitation on the display mode of the present liquid crystal display device, and it is possible to use the invention for all the transmissive and reflective liquid crystal display devices. Among these, the invention exerts the effect for the VA mode to which the improvement in color viewing angle property is desired.

An example of the liquid crystal display device of the present invention is a VA-mode liquid crystal display device. Examples of the VA-mode liquid crystal display device include those employing a negative C-plate and A-plate for optical compensation, and those employing a single biaxial film for optical compensation. The optically anisotropic film of the present invention may be used in any of both embodiments, wherein it may be used as an A-plate in the former, and as a biaxial film for the latter. In the former embodiment, the negative C-plate used in combination with the optically anisotropic film of the present invention is generally understood as having optical characteristics satisfying nx=ny>nz. The negative C-plate used for optical compensation of the VA-mode liquid crystal display device preferably has Rth(550) of 20 to 300 nm, more preferably 50 to 250 nm, and still more preferably 100 to 250 nm. The negative C-plate may be composed of any material. Any of birefringent polymer films, or optically anisotropic layers formed using curable liquid crystal compositions may be usable. More specifically, examples of which include birefringent films obtained by stretching films composed of appropriate polymers such as polycarbonate, norbornene resins, polyvinyl alcohol, polystyrene, polymethylmethacrylate, polypropylene, polyolefin, polyarylate and polyamide; alignment film composed of liquid crystal compound such as liquid crystal polymer; and stack having an alignment layer composed of liquid crystal material formed on a support. Birefringent films obtained by biaxial stretching or by stretching in two directions normal to each other, and biaxially stretched film such as inclined alignment film may be usable. The inclined alignment film may be exemplified by those obtained by bonding a polymer film with a heat-shrinkable film, and by stretching or/and shrinking the polymer film under force of shrinkage induced by heating, and those obtained by allowing the liquid crystal polymer to obliquely align.

Although the description in the above dealt with the embodiments of the VA-mode liquid crystal display device, the optically anisotropic film of the present invention may be applicable also to optical compensation of other modes of liquid crystal display devices. Retardation plates (optical compensation sheets) for the TN-mode liquid crystal cell are described in JPA No. H6-214116, U.S. Pat. Nos. 5,583,679 and 5,646,703 and German Patent No. 3911620A1. A retardation plate (optical compensation sheet) for IPS-mode or FLC-mode liquid crystal cell is described in JPA No. H10-54982. Alternatively, retardation plates (optical compensation sheets) for the OCB-mode or HAN-mode liquid crystal cell are described in U.S. Pat. No. 5,805,253 and the pamphlet of WO96/37804. Still alternatively, a retardation plate (optical compensation sheet) for the STN-mode liquid crystal cell is described in Japanese Laid-Open Patent Publication No. H9-26572. A retardation plate (optical compensation sheet) for the VA-mode liquid crystal cell is described in Japanese Patent No. 2866372. The optically anisotropic film of the present invention may be applicable also as a substitute of these optical compensation sheets.

Use of the optically anisotropic film of the present invention, in combination with polarizer plate(s), is also effective for the purpose of anti-reflection of electro-luminescence devices and field emission display devices.

### EXAMPLES

The invention is described more concretely with reference to the following Examples, in which the material and the reagent used, their amount and the ratio, the details of the treatment and the treatment process may be suitably modified or changed not overstepping the sprit and the scope of the invention. Accordingly, the invention should not be limited by the Examples mentioned below.

### [Exemplary Synthesis 1: Synthesis of Compound A-1]

Under the presence 16.5 g of potassium carbonate and 22.5 mg of palladium acetate, 23.4 g of 4-bromo-4′-hydroxybiphenyl and 15.05 g of n-butyl acrylate were allowed to react in 75 ml of dimethylacetamide at 130° C. After the reaction, the mixture was diluted with ethyl acetate, the ethyl acetate phase was washed with water, and then purified by column chromatography, to thereby obtain Compound C-1.

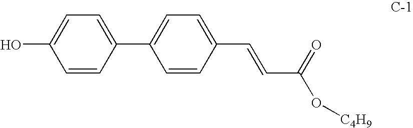

Dissolved was 2.53 g of Compound C-2, shown below, synthesized according to a publicly-known synthetic method into tetrahydrofuran, and the solution was cooled to 5° C. The solution was added dropwise with 1.15 g of methanesulfonyl chloride and 1.30 g of diisopropyl ethylamine, the mixture was stirred at room temperature for 1 hour and 30 minutes, and then cooled again to 5° C. The mixture was added with 2.70 g of Compound C-1, 1.30 g of diisopropyl ethylamine, and 0.13 g of 4-dimethylaminopyridine were added. The mixture was stirred at room temperature for 1 hour and 30 minutes, and then the reaction solution was diluted with ethyl acetate, and washed with water. Solid content of the ethyl acetate phase was purified by column chromatography, to thereby obtain Compound A-1 exemplified above.

The identification of the compound was performed by NMR.

¹H-NMR (CDCl₃, ppm) of a compound A-1: 0.9-1.1 1.3-1.8, 1.8-2.1, 4.0-4.4, 5.7-6.6, 6.9-7.1, 7.2-7.4, 7.5-7.8, and 8.1-8.3.

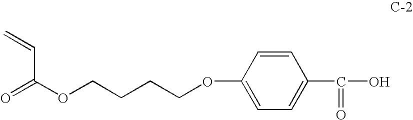

### [Synthesis 2: Synthesis of Compound P-1]

Compound A-1 synthesized in the above and Compound B-2 were polymerized, under presence of azoisobutyronitrile (AIBN), in dimethylacetamide, to thereby obtain Compound P-1. The weight-average molecular weight of P-1 was found to be 45,000.

### Example 1

### Fabrication of Substrate for Composing Liquid Crystal Cell

A substrate composed of non-alkali glass and having a black matrix formed thereon was prepared.

### (Preparation of Coating Liquid LC-1 for Forming Optically Anisotropic Layer)

The composition below was prepared, filtered through a polypropylene filter having a pore size of 0.2 μm, and the filtrate was used as a coating liquid LC-1 for forming the optically anisotropic layer.

LC-1-1 was synthesized according to the method described in Tetrahedron Lett., Vol. 43, p. 6793 (2002).

Formulation of Coating Liquid for Forming Optically
Anisotropic layer (% by mass)

| | |
|---|---|
| P-1 having weight-average molecular weight of 45,000 | 25.0 |
| 1.4-Butanediol diacetate | 74.98 |
| Horizontal-alignment aid (LC-1-1) | 0.02 |

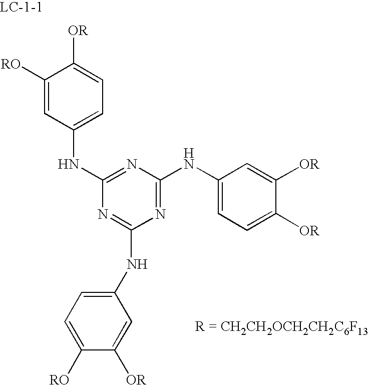

### (Composition for Forming Color Filter)

Compositions for forming the R, G and B layers, having formulations listed in Table 2, were respectively prepared.

TABLE 2
% by mass

| | PP-R1 | PP-G1 | PP-B1 |
|---|---|---|---|
| R pigment dispersion-1 | 44 | — | — |
| R pigment dispersion-2 | 5.0 | — | — |
| G pigment dispersion | — | 24 | — |
| CF Yellow EC3393 | — | 13 | — |
| (from Mikuni Color Works, Ltd.) | | | |
| CF Blue EX3357 | — | — | 7.2 |
| (from Mikuni Color Works, Ltd.) | | | |
| CF Blue EX3383 | — | — | 13 |
| (from Mikuni Color Works, Ltd.) | | | |
| propylene glycol monomethyl ether | 76 | 29 | 23 |
| acetate (PGMEA) | | | |
| methyl ethyl ketone | 37.412 | 25.115 | 35.78 |
| Cyclohexanone | — | 1.3 | — |
| binder 1 | — | 2.9 | — |
| binder 2 | 0.7 | — | — |
| binder 3 | — | — | 16.9 |
| DPHA solution | 4.4 | 4.3 | 3.8 |
| 2-trichloromethyl-5-(p-styrylmethyl)- | 0.14 | 0.15 | 0.15 |
| 1,3,4-oxadiazole | | | |
| 2,4-bis(trichloromethyl)-6-[4-(N,N- | 0.058 | 0.060 | — |
| diethoxycarbonylmethyl)-3-bromophenyl]-s- | | | |
| triazine | | | |
| Phenothiazine | 0.010 | 0.005 | 0.020 |
| hydroquionone monomethyl ether | — | — | — |
| Hexafluoro antimonic acid triallyl | 3.37 | 2.00 | 2.00 |
| sulfonium | | | |
| HIPLAAD ED152 (from Kusumoto | 0.52 | — | — |
| Chemicals) | | | |
| Megafac F-176PF (from Dainippon Ink | 0.060 | 0.070 | 0.050 |
| and Chemicals, Inc.) | | | |

The formulations of the compositions listed in Table 2 are as follows.

### [Formulation R Pigment Dispersion-1]

| Formulation of R Pigment Dispersion-1 | (% by mass) |
|---|---|
| C.I. Pigment Red 254 | 8.0 |
| 5-[3-oxo-2-[4-[3,5-bis(3-diethyl aminopropyl | 0.8 |
| aminocarbonyl)phenyl]aminocarbonyl]phenylazo]- | |
| butyroylaminobenzimidazolone | |
| random copolymer of benzyl methacrylate/methacrylic | 8.0 |
| acid (72/28 by molar ratio, weight-average molecular | |
| weight = 37,000) | |
| propylene glycol monomethyl ether acetate | 83.2 |

### [Formulation of R Pigment Dispersion-2]

| Formulation of R Pigment Dispersion-2 | (% by mass) |
|---|---|
| C.I. Pigment Red 177 | 18.0 |
| random copolymer of benzyl methacrylate/methacrylic | 12.0 |
| acid (72/28 by molar ratio, weight-average molecular | |
| weight = 37,000) | |
| propylene glycol monomethyl ether acetate | 70.0 |

### [Formulation of G Pigment Dispersion]

| Formulation of G Pigment Dispersion | (% by mass) |
|---|---|
| C.I. Pigment Green 36 | 18.0 |
| random copolymer of benzyl methacrylate/methacrylic | 12.0 |
| acid (72/28 by molar ratio, weight-average molecular | |
| weight = 37,000) | |
| cyclohexanone | 35.0 |
| propylene glycol monomethyl ether acetate | 35.0 |

### [Formulation of Binder 1]

| Formulation of Binder 1 | (% by mass) |
|---|---|
| random copolymer of benzyl methacrylate/methacrylic | 27.0 |
| acid (78/22 by molar ratio, weight-average molecular | |
| weight = 40,000) | |
| propylene glycol monomethyl ether acetate | 73.0 |

### [Formulation of Binder 2]

| Formulation of Binder 2 | (% by mass) |
|---|---|
| random copolymer of benzyl methacrylate/methacrylic | 27.0 |
| acid/methyl methacrylate (38/25/37 by molar ratio, | |
| weight-average molecular weight = 30,000) | |
| propylene glycol monomethyl ether acetate | 73.0 |

### [Formulation of Binder 3]

| Formulation of Binder 3 | (% by mass) |
|---|---|
| random copolymer of benzyl methacrylate/methacrylic | 27.0 |
| acid/methyl methacrylate (36/22/42 by molar ratio, | |
| weight-average molecular weight = 30,000) | |
| propylene glycol monomethyl ether acetate | 73.0 |

### [Formulation of DPHA]

| Formulation of DPHA Solution | (% by mass) |
|---|---|
| KAYARAD DPHA (from Nippon Kayaku Co., Ltd.) | 76.0 |
| propylene glycol monomethyl ether acetate | 24.0 |

### (Preparation of R-Layer-Forming Liquid PP-R1)

First, R pigment dispersion 1, R pigment dispersion 2 and propylene glycol monomethylether acetate were weighed to as much as being listed in Table 2, mixed at 24° C. (±2° C.), and the mixture was stirred at 150 rpm for 10 minutes. Next, methyl ethyl ketone, binder 2, DPHA solution, 2-trichloro methyl-5-(p-styrylmethyl)-1,3,4-oxadiazole, 2,4-bis(trichloromethyl)-6-[4-(N,N-diethoxy carbonylmethyl)-3-bromophenyl]-s-triazine, and phenothiazine were weighed to as much as being listed in Table 2, added in this order to the above mixture at 24° C. (±2° C.), and the mixture was stirred at 150 rpm for 10 minutes. Next, ED152 was weighed to as much as being listed in Table 2, and was added to the mixture at 24° C. (±2° C.), the mixture was stirred at 150 rpm for 20 minutes, Megafac F-176 PF was weighed to as much as being listed in Table 2, and added to the mixture at 24° C. (±2° C.), the mixture was stirred at 30 rpm for 30 minutes, and then filtered through a nylon mesh #200, to thereby obtain an R-layer-forming liquid PP-R1.

### (Preparation of Liquid for Forming G-Layer-Forming Liquid PP-G1)

First, G pigment dispersion, CF Yellow EX3393, and propylene glycol monomethylether acetate were weighed to as much as being listed in Table 2, mixed at 24° C. (±2° C.), and the mixture was stirred at 150 rpm for 10 minutes. Next, methyl ethyl ketone, cyclohexanone, binder 1, DPHA solution, 2-trichloromethyl-5-p-styrylmethyl)-1,3,4-oxadiazole, 2,4-bis(trichloromethyl)-6-[4-(N,N-diethoxycarbonylmethyl)-3-bromophenyl]-s-triazine, and phenothiazine were weighed to as much as being listed in Table 2, added in this order to the above mixture 24° C. (±2° C.), and the mixture was stirred at 150 rpm for 30 minutes. Megafac F-176 PF was then weighed to as much as being listed in Table 2, added to the mixture at 24° C. (±2° C.), the mixture was stirred at 30 rpm for 5 minutes, and filtered through a nylon mesh #200, to thereby obtain a G-layer-forming liquid PP-G1.

### (Preparation of B-Layer-Forming Liquid PP-B1)

First, CF Blue EX3357, CF Blue EX3383, and propylene glycol monomethylether acetate were weight to as much as being listed in Table 2, mixed at 24° C. (±2° C.), and the mixture was stirred at 150 rpm for 10 minutes. Next, methyl ethyl ketone, binder 3, DPHA solution, 2-trichloromethyl-5-(p-styrylmethyl)-1,3,4-oxadiazole, and phenothiazine were weighed to as much as being listed in Table 2, added to the mixture in this order at 25° C. (±2° C.), and the mixture was stirred at 40° C. (±2° C.) and at 150 rpm for 30 minutes. Megafac F-176 PF was then weighed to as much as being listed in Table 2, added to the mixture at 24° C. (±2° C.), the mixture was stirred at 30 rpm for 5 minutes, and filtered through a nylon mesh #200, to thereby obtain a B-layer-forming liquid PP-B1.

### (Fabrication of Optically Anisotropic Layer)

Coating liquid LC-1 for forming optically anisotropic layer was applied using a piezoelectric head to recesses surrounded by the black matrix (light-shielding partition) destined for formation of an R layer, and dried at 140° C. for 2 minutes. The layer was further matured, then irradiated with polarized UV (illumination intensity=200 mW/cm², and energy of irradiation=200 mJ/cm²), and matured under heating at 130° C., to thereby form an optically anisotropic layer, R-1, for R color to as thick as 2.8 μm.

Similarly, Optically anisotropic layers G-1 and B-1 for G color and B color were respectively formed in fine regions destined for formation of G layer and B layer. Thickness of each of Optically anisotropic layers G-1 and B-1 was adjusted to 2.9 μm and 2.6 μm, respectively, by varying the amount of spotting.

In this Example, the coating liquid for forming optically anisotropic layer was applied to the desired recesses corresponded to R, G and B colors in each pixel, by controlling carrying speed and operation frequency.

### (Fabrication of Color Filter Layers)

The coating liquids PP-R1, PP-G1 and PP-B1 for forming R, G and B layers obtained in the above were applied using a piezoelectric head to the recesses surrounded by the light-shielding partition at predetermined positions, to thereby form the R layer, G layer and B layer, respectively.

In this Example, the coating liquids PP-R1, PP-G1 and PP-B1 for forming R, G and B layers were applied to the desired recesses corresponded to R, G and B colors in each pixel, by controlling carrying speed and operation frequency as being respectively adapted to R, G and B colors.

The coating liquids were dried at 100° C., further annealed at 200° C. for one hour, to thereby form color filter pixels on the optically anisotropic layer.

### (Measurement of Retardation)

According to a parallel Nicol method employing a fiber spectrophotometer, retardation in plane Re(λ) at arbitrary wavelength λ of the sample, and retardation of the sample measured for light in the direction rotated ±40° C. relative to the normal line direction, while assuming the slow axis as the axis of rotation, were respectively measured, and using the obtained values, Rth(λ) and Nz value were determined. Retardation was measured at 611 nm, 545 nm and 435 nm for R, G and B, respectively.

Retardation of the optically anisotropic layer was obtained as a value solely ascribable thereto, by correcting the measured value using a preliminarily-obtained data of transmissivity of the substrate having no optically anisotropic layer formed thereon. Results of measurement of retardation are shown in the following table. It may be understood from the values in the table, that thus-formed Optically anisotropic layers R-1, G-1 and B-1 are biaxial.

| Optically anisotropic layer | Re (λ) | Nz value |
|---|---|---|
| R-1 | 46 nm | 3.2 |
| G-1 | 49 nm | 3.9 |
| B-1 | 34 nm | 5.6 |

### (Formation of Transparent Electrode)

A transparent electrode film (200 nm thick) of ITO was formed on the color filter fabricated in the above according to a sputtering method.

### (Formation of Alignment Layer and Liquid Crystal Cell)

An alignment film made of polyimide was provided further thereon. Next, glass bead of 5 μm in diameter was dusted. An epoxy resin sealant containing spacer particles was then printed at the position corresponded to the outer frame of the black matrix provided around the pixel group of color filter, and the color filter substrate and the opposing substrate were bonded under a pressure of 10 kg/cm². Thus bonded glass substrates were then annealed at 150° C. for 90 minutes so as to cure the sealant, to thereby obtain a stack of two glass substrates. The stack of glass substrates was evacuated in vacuo, and a liquid crystal was injected into a gap between two glass substrate by recovering therein the atmospheric pressure, to thereby obtain a liquid crystal cell. Polarizer plates HLC2-2518 from Sanritz Corporation were bonded on both surfaces of the liquid crystal cell.

### (Fabrication of VA-LCD)

As a cold cathode ray tube, which is used for a backlight of color liquid crystal display device, a white three-wavelength fluorescent lamp having an arbitrary hue was fabricated using a 50:50 (w/w) mixed phosphor of BaMg₂Al₁₆O₂₇:Eu,Mn and LaPO₄:Ce,Tb for green (G), Y₂O₃:Eu for red (R), and BaMgAl₁₀O₁₇:Eu for blue (B). Over the backlight, the liquid crystal cell provided with the polarizer plate was disposed, to thereby fabricate a VA-LCD.

### Example 2

A VA-LCD of Example 2 was fabricated similarly to as in Example 1, except that Optically anisotropic layers R-1, G-1, B-1 were omitted, and instead an optically anisotropic layer of 2.7 μm thick was formed using Coating liquid LC-1 on a protective film to be disposed the liquid crystal cell side of the lower polarizer plate, by a similar method by which the optically anisotropic layer G-1 was fabricated.

### [Evaluation of VA-LCDs in Examples 1 and 2]

Quality of the black state of each of the fabricated liquid crystal display devices was observed at a viewing angle expressed by an azimuth angle of 45° and a polar angle of 60°, and color shift between a viewing angle expressed by an azimuth angle of 45° and a polar angle of 60°, and a viewing angle expressed by an azimuth angle of 180° and a polar angle of 60° was observed.

It was confirmed from the observation of the fabricated liquid crystal display devices of Examples 1 and 2, that neutral black state was achieved both in the normal line direction and in the oblique direction. In particular, Example 1 was found to completely be suppressed in coloration in the oblique direction, and proved its excellence.

### Example 3

### Preparation of Coating Liquid LC-2 for Forming Optically Anisotropic Layer

Coating liquid LC-2 for forming the optically anisotropic layer was prepared similarly to Coating liquid LC-1 for forming the optically anisotropic layer of Example 1, except that Compound P-1 having a weight-average molecular weight of 15,000 was used in place of Compound P-1 having a weight-average molecular weight of 45,000 used in Example 1.

### (Fabrication of Optically Anisotropic Layer)

Similarly to as described in Example 1, the coating liquid LC-2 for forming the optically anisotropic layer obtained in the above was applied using a piezoelectric head to the recesses corresponded to the R, G and B layers surrounded by the light-shielding partition, and was then dried under heating at 140° C. for 2 minutes. The layer was irradiated with polarized light (illumination intensity=200 mW/cm², energy of irradiation=200 mJ/cm²), the heated again at 130° C., to thereby form Optically anisotropic layers R-2, G-2 and B-2.

The color filter layers and a transparent electrode layer were then formed, similarly to as described in Example 1.

Further thereon, a polyimide alignment film was provided. Next, a glass bead of 5 μm in diameter was dusted. An epoxy resin sealant containing spacer particles was then printed at the position corresponded to the outer frame of the black matrix provided around the pixel group of color filter, and the color filter substrate and the opposing substrate were bonded under a pressure of 10 kg/cm². Thus bonded glass substrates were then annealed at 150° C. for 90 minutes so as to cure the sealant, to thereby obtain a stack of two glass substrates. The stack of glass substrates was evacuated in vacuo, and a liquid crystal was injected into a gap between two glass substrate by recovering therein the atmospheric pressure, to thereby obtain a liquid crystal cell.

A polarizing plate having a negative C-plate formed thereon, fabricated by a method described in JPA No. 2005-173567, was used as the upper polarizing plate (on the observer's side) of the liquid crystal cell. A polarizing plate HLC2-2518 from Sanritz Corporation was used as the lower polarizing plate (on the backlight side). The negative C-plate was found to show Re of 0 nm, and Rth of 200 nm.

### (Fabrication of VA-LCD)

As a cold cathode ray tube, which is used for a backlight of color liquid crystal display device, a white three-wavelength fluorescent lamp having an arbitrary hue was fabricated using a 50:50 (w/w) mixed phosphor of BaMg₂Al₁₆O₂₇:Eu,Mn and LaPO₄:Ce,Tb for green (G), Y₂O₃:Eu for red (R), and BaMgAl₁₀O₁₇:Eu for blue (B). Over the backlight, the liquid crystal cell provided with the polarizing plate was disposed, to thereby fabricate a VA-LCD.

### (Measurement of Retardation)

Retardation in plane of each of thus-fabricated Optically anisotropic layers R-2, G-2 and B-2 was measured similarly to as described in the above. Results are shown in the following table. It may be understood from the values in the table, that Optically anisotropic layers R-2, G-2 and B-2 are A-plate-like.

| Optically anisotropic layer | Re (λ) | Nz value |
|---|---|---|
| R-2 | 131 nm | 1.0 |
| G-2 | 128 nm | 1.0 |
| B-2 | 120 nm | 1.0 |

### Example 4

A VA-LCD of Example 4 was fabricated similarly to as in Example 3, except that Optically anisotropic layers R-2, G-2, B-2 were omitted, and instead Optically anisotropic layer G-2 was formed using Coating liquid LC-2 on a protective film to be disposed on the liquid crystal cell side of the lower polarizer plate, according to a similar method by which Optically anisotropic layer G-2 was fabricated.

### [Evaluation of VA-LCDs of Examples 3 and 4]

Quality of the black state of each of the fabricated liquid crystal display devices was observed at a viewing angle expressed by an azimuth angle of 45° and a polar angle of 60°, and color shift between a viewing angle expressed by an azimuth angle of 45° and a polar angle of 60°, and a viewing angle expressed by an azimuth angle of 180° and a polar angle of 60° was observed.

It was confirmed from the observation of fabricated liquid crystal display devices of Examples 3 and 4, that neutral black state was achieved both in the normal line direction and in the oblique direction. In particular, Example 3 completely suppressed in coloration in the oblique direction proved its excellence.

### Example 5

Coating liquid LC-4 for forming the optically anisotropic layer was prepared similarly to as described in Example 2, except that Coating liquid LC-2 for forming the optically anisotropic layer in Example 3 was added with a cationic photo-polymerization initiator, and that Compound P-6 was used in place of P-1, as a photo-alignable polymer material. The formulation is shown below.

| | | |
|---|---|---|
| Compound P-6 exemplified above | 25.0% | by mass |
| 1,4-Butanediol diacetate | 74.58% | by mass |
| Horizontal alignment aid (LC-1-1) | 0.02% | by mass |
| Cationic photo-polymerization initiator | 0.40% | by mass |
| (Cyracure UVI6974, from Dow Chemical Company) | | |

Similarly to as described in Example 1, Coating liquid LC-4 for forming the optically anisotropic layer obtained in the above was applied using a piezoelectric head to the recesses corresponded to the R layer surrounded by the light-shielding partition, and was then dried under heating at 140° C. for 2 minutes. The layer was further matured, and immediately thereafter, irradiated with polarized UV (illumination intensity=200 mW/cm², energy of irradiation=200 mJ/cm²), matured under heating at 130° C., and then cured by irradiation of non-polarized UV, to thereby form Optically anisotropic layer R-4 of 2.6 μm thick.

Similarly, Optically anisotropic layers G-4 and B-4 for the G layer and the B layer were respectively formed in the fine regions destined for formation of the G layer and the B layer. Thickness of each of Optically anisotropic layers G-4 and B-4 was adjusted to 2.7 μm and 2.9 μm, respectively, by varying the amount of spotting.

Thereafter, the color filter layers were formed similarly to as described in Example 1, and then annealed at 230° C. Thereafter, similarly to as described in Example 1, retardation in plane of optically anisotropic layers R-4, G-4 and B-4 were measured. Results are shown in the following table.

| Optically anisotropic layer | Re (λ) |
|---|---|
| R-3 | 130 nm |
| G-3 | 128 nm |
| B-4 | 116 nm |

### Comparative Example 1

The optically anisotropic layer was fabricated similarly to as described in Example 1, except that Coating liquid LC-4 of Example 5 was prepared using, in place of P-6, a compound described in JPA No. 2004-258426. The annealing after formation of the color filter was carried out at 230° C., and retardation of the optically anisotropic layer was measured, only to find that retardation had disappeared.

### Example 6

### Preparation of Coating Liquid AL-1 for Forming Alignment Layer

The composition shown below was prepared, filtered through a polypropylene filter having a pore size of 30 μm, and the filtrate was used as Coating liquid AL-1 for forming the alignment layer.

| Composition of Coating Liquid for Forming Alignment | |
|---|---|
| Layer | (% by mass) |
| Polyvinyl alcohol (PVA205, from Kuraray Co., Ltd.) | 3.21 |
| Polyvinyl pyrrolidone (Luvitec K30, from BASF) | 1.48 |
| Distilled water | 52.1 |
| Methanol | 43.21 |

### (Fabrication of Alignment Layer)

Coating liquid AL-1 for forming the alignment layer obtained in the above was applied using a piezoelectric head to the recesses surrounded by the light-shielding partition, and dried. The alignment layer was as thick as 1.6 μm. Thus-formed alignment layer was then rubbed at an angle of 45° relative to the transverse direction thereof assumed as 0°.

### (Fabrication of Optically Anisotropic Layer)

Coating liquid LC-5 for forming the optically anisotropic layer was prepared similarly to as described in Example 1, except that Compound P-1 used for preparing Coating liquid LC-1 for forming the optically anisotropic layer was replaced with the exemplary compound P-16.

Similarly to as described in Example 1, Coating liquid LC-5 for forming the optically anisotropic layer obtained in the above was applied using the piezoelectric head to the recesses corresponded to the R, G and B layers surrounded by the light-shielding partition, and dried under heating at 140° C. for 2 minutes. Immediately thereafter, the layer was irradiated with polarized light in the transverse direction (illumination intensity=200 mW/cm², energy of irradiation=200 mJ/cm²), and heated again at 130° C. to thereby form Optically anisotropic layers R-5, G-5, and B-5.

The color filter layers and a transparent electrode layer were then formed, similarly to as described in Example 1.

Further thereon, a polyimide alignment film was provided. Next, a glass bead of 5 μm in diameter was dusted. An epoxy resin sealant containing spacer particles was then printed at the position corresponded to the outer frame of the black matrix provided around the pixel group of color filter, and the color filter substrate and the opposing substrate were bonded under a pressure of 10 kg/cm². Thus bonded glass substrates were then annealed at 150° C. for 90 minutes so as to cure the sealant, to thereby obtain a stack of two glass substrates. The stack of glass substrates was evacuated in vacuo, and a liquid crystal was injected into a gap between two glass substrate by recovering therein the atmospheric pressure, to thereby obtain a liquid crystal cell. On both surfaces of the liquid crystal cell, polarizing plates HLC2-2518 from Sanritz Corporation were bonded.

### (Fabrication of VA-LCD)

As a cold cathode ray tube, which is used for a backlight of color liquid crystal display device, a white three-wavelength fluorescent lamp having an arbitrary hue was fabricated using a 50:50 (w/w) mixed phosphor of BaMg₂Al₁₆O₂₇:Eu,Mn and LaPO₄:Ce,Tb for green (G), Y₂O₃:Eu for red (R), and BaMgAl₁₀O₁₇:Eu for blue (B). Over the backlight, the liquid crystal cell provided with the polarizer plate was disposed, to thereby fabricate a VA-LCD.

### (Measurement of Retardation)

Retardation in plane of each of Optically anisotropic layers R-5, G-5 and B-5 was measured similarly to as described in the above. Results of measurement of are shown in the following table. It may be understood from the values in the table, that Optically anisotropic layers R-5, G-5 and B-5 are biaxial.

| | Re | Nz value |
|---|---|---|
| R-5 | 45 nm | 3.1 |
| G-5 | 48 nm | 3.8 |
| B-5 | 33 nm | 5.5 |

### [Evaluation of VA-LCD of Example 6]

Quality of the black state of each of the fabricated liquid crystal display device was observed at a viewing angle expressed by an azimuth angle of 45° and a polar angle of 60°, and color shift between a viewing angle expressed by an azimuth angle of 45° and a polar angle of 60°, and a viewing angle expressed by an azimuth angle of 180° and a polar angle of 60° was observed.

It was confirmed from the observation of thus fabricated liquid crystal display device of Example 6, that neutral black state was achieved both in the normal line direction and in the oblique direction.

### Example 7

### Fabrication of Optically Anisotropic Layer

Coating liquid LC-3 for forming optically anisotropic layer was prepared similarly to as described in Example 1, except that Compound P-1 used for preparing Coating liquid LC-1 for forming the optically anisotropic layer in Example 1 was replaced with Compound P-2.

Similarly to as described in Example 1, the coating liquid LC-3 for forming the optically anisotropic layer obtained in the above was applied using the piezoelectric head to the recesses corresponded to the R, G and B layers surrounded by the light-shielding partition, and dried under heating at 140° C. for 2 minutes. Immediately thereafter, the layer was irradiated with polarized light from the transverse direction (illumination intensity=200 mW/cm², energy of irradiation=200 mJ/cm²), and heated again at 130° C. to thereby form the optically anisotropic layers R-3, G-3, B-3.

### (Measurement of Retardation)

Retardation in plane of each of Optically anisotropic layers R-3, G-3 and B-3 was measured similarly to as described in the above. Results of measurement of are shown in the following table. It may be understood from the values in the table, that Optically anisotropic layers R-3, G-3 and B-3 satisfy nx>nz>ny.

| | Re | Nz Value |
|---|---|---|
| R-3 | 305 nm | 0.49 |
| G-3 | 272 nm | 0.5 |
| B-3 | 217 nm | 0.51 |

### (Fabrication of IPS-Mode Liquid Crystal Cell)

A polyimide film was provided on one surface of the glass substrate, and the rubbed to thereby form an alignment film.

On one separately-obtained glass substrate, and in the region thereof where the pixels of a liquid crystal element will be formed, electrodes were provided while keeping a 20 μm distance between every adjacent electrodes, a polyimide film was provided thereon as an alignment film, and rubbed. Two these glass substrates were held so as to oppose the alignment films with each other, and then bonded while keeping a distance (gap; d) of 3.9 μm in between, and while aligning the rubbing direction of two glass substrate in parallel with each other. A nematic liquid crystal composition having a refractive index anisotropy (Δn) of 0.0769 and a dielectric anisotropy (λε) of +4.5 was filled in the gap. The liquid crystal layer was found to show a d·Δn value of 300 nm.

### (Fabrication of Optically Anisotropic Film B-1)

### <Preparation of Cellulose Acetate Solution>

The ingredients below were placed in a mixing tank, and stirred to dissolve the individual ingredients, to thereby prepare Cellulose acetate solution “A”.

### <Formulation of Cellulose Acetate Solution “A”>

| | | |
|---|---|---|
| Cellulose acetate having a degree of acetylation | 100.0 | parts by mass |
| of 2.86 | | |
| Methylene chloride (first solvent) | 402.0 | parts by mass |
| Methanol (second solvent) | 60.0 | parts by mass |

### <Preparation of Matting Agent Dispersion>

Twenty parts by mass of silica particle having a mean particle size of 16 nm (AEROSIL R972, from Nippon Aerosil Co., Ltd.) and 80 parts by mass of methanol were thoroughly mixed under stirring, to thereby prepare a silica particle dispersion. The dispersion was placed in a disperser together with the ingredients below, and further stirred for 30 minutes or longer so as to dissolve the individual ingredients, to thereby prepare a matting agent dispersion.

### <Formulation of Matting Agent Dispersion>

| | | |
|---|---|---|
| Dispersion of silica particle having mean particle | 10.0 | parts by mass |
| size of 16 nm | | |
| Methylene chloride (first solvent) | 76.3 | parts by mass |
| Methanol (second solvent) | 3.4 | parts by mass |
| Cellulose acetate solution “A” | 10.3 | parts by mass |

The composition below was placed into a mixing tank, stirred under heating so as to dissolve the individual ingredients, to thereby prepare a cellulose acetate solution.

### <Composition of Additive Solution>

| | | |
|---|---|---|
| Optical anisotropy reducing compound (A-01) | 49.3 | parts by mass |
| Chromatic dispersion adjusting agent (UV-01) | 7.6 | parts by mass |
| Methylene chloride (first solvent) | 58.4 | parts by mass |
| Methanol (second solvent) | 8.7 | parts by mass |
| Cellulose acetate solution “A” | 12.8 | parts by mass |

LogP value of A-01 was found to be 2.9.

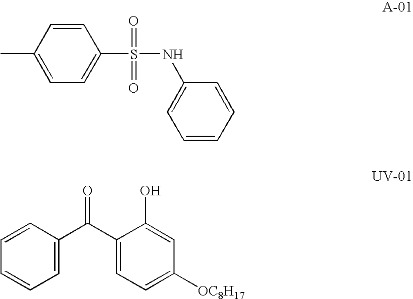

### <Fabrication of Cellulose Acetate Film>

A dope was prepared by mixing 94.6 parts by mass of Cellulose acetate solution “A”, 1.3 parts by mass of the matting agent dispersion, and 4.1 parts by mass of the additive solution, after being independently filtered. In this dope, ratios by weight of the optical anisotropy reducing compound and the chromatic dispersion adjusting agent relative to cellulose acetate were found to be 12% and 1.8%, respectively.

The dope was cast on a band using a casting machine, the resultant film in the state of keeping a residual solvent content of 30% was peeled off from the band, and dried at 140° C. for 40 minutes, to thereby obtain a cellulose acetate film. The obtained cellulose acetate film was found to have a residual solvent content of 0.2%, and a thickness of 40 μm.

The film was also found to have a Re(630) of 0.3 nm, a Rth (630) of 3.2 nm, a |Re(400)-Re(700)| of 1.2 nm, a |Rth(400)-Rth(700)| of 7.5 nm, a Tg of the film of 134.3° C., a haze of film of 0.34%, and a ΔRth (10% RH-80% RH) of 24.9 nm. The film was named as Optically anisotropic film B-1.

(Fabrication of Polarizing Plate 1 with Optically Anisotropic Film B-1)

A stretched polyvinyl alcohol film was allowed to adsorb iodine to thereby fabricate a polarizing film. A commercially-available cellulose acetate film (Fujitac TD80UF, from FIJJIFILM Corporation) was saponified and bonded to one surface of the polarizing film, and Optically anisotropic film B-1 was bonded on the other surface, using a polyvinyl alcohol-base adhesive, to thereby form Polarizing plate 1.

### (Fabrication of IPS-LCD)

Polarizing plate 1 was bonded on one side of the IPS-mode liquid crystal cell fabricated in the above, so as to align the transmission axis of the polarizing film in parallel with the direction of rubbing of the liquid crystal cell, and Polarizing plate 1 was bonded to the other side of the IPS-mode liquid crystal cell, to thereby fabricate a liquid crystal display device.

Quality of the black state of the fabricated liquid crystal display device was observed at a viewing angle expressed by an azimuth angle of 45° and a polar angle of 60°, and color shift between a viewing angle expressed by an azimuth angle of 45° and a polar angle of 60°, and a viewing angle expressed by an azimuth angle of 180° and a polar angle of 60° was observed.

It was confirmed from the observation of the fabricated IPS-LCD of Example 7, that neutral black state was achieved both in the normal line direction and in the oblique direction.

What is claimed is:

1. An optically anisotropic film comprising at least one compound having a partial structure represented by formula (1) below: 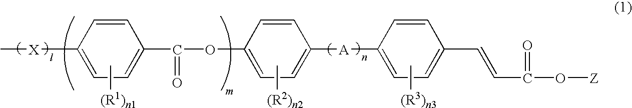
where, each of R¹, R² and R³ independently represent a substituent; X represents a divalent linking group selected from Linking Group I shown below, or a divalent linking group formed by combining two or more species selected from Linking Group I shown below; “A” represents —COO—, —OCO—, or a substituted or non-substituted phenylene group, oxadiazole group or alkynylene group; Z represents a substituted or non-substituted alkyl group or aryl group; each of n1, n2 and n3 represents an integer of 0 to 4; and each of l, m and n represents an integer of 0 to 4;
Linking Group I:
single bond, —O—, —CO—, —NR⁶— (R⁶ represents a hydrogen atom, alkyl group or aryl group), —S—, —SO₂—, —P(═O)(OR⁷)— (R⁷ represents an alkyl group or aryl group), alkylene group and arylene group.

2. The optically anisotropic film of claim 1, wherein said compound is a polymer compound having the partial structure represented by formula (1) in the side chain(s) thereof.

3. The optically anisotropic film of claim 2, wherein said polymer compound comprises a repeating unit represented by formula (2): 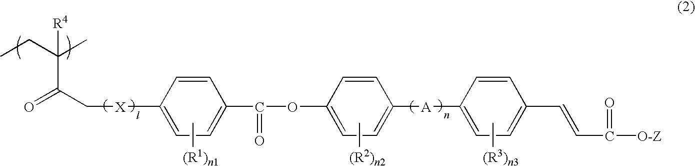
where, R⁴ represents a hydrogen atom or substituent, and other symbols are used for the same meaning with those in formula (1).

4. The optically anisotropic film of claim 2, wherein said polymer compound further comprises a repeating unit represented by formula (5) and/or formula (7) below: 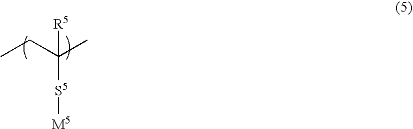
where, R⁵ represents a hydrogen atom or substituent, S⁵ represents a divalent linking group, and M⁵ represents a mesogen group; 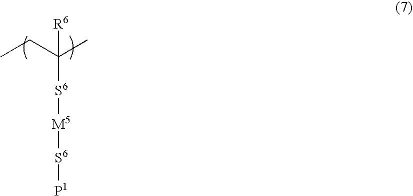
where, R⁵ represents a hydrogen atom or substituent, S⁵ represents a divalent linking group, M⁵ represents a mesogen group, S⁶ represents a divalent linking group, and P¹ represents a polymerizable group.

5. The optically anisotropic film of claim 1, formed of a composition comprising at least said compound irradiated with polarized light.

6. The optically anisotropic film of claim 5, having Re(550), which is retardation in plane at 550 nm, is 20 nm to 300 nm.

7. The optically anisotropic film of claim 5, being a positive A-plate.

8. The optically anisotropic film of claim 5, having an Nz value, where Nz=Rth(550)/Re(550)+0.5, Rth(550) is retardation along thickness direction at 550 nm, and Re(550) is retardation in plane at 550 nm, of 1.1 to 7.0.

9. The optically anisotropic film of claim 1, formed of a composition comprising at least said compound irradiated with polarized light on a rubbed surface.

10. The optically anisotropic film of claim 9, having an Nz value, where Nz-Rth(550)/Re(550)+0.5, Rth(550) is retardation along thickness direction at 550 nm, and Re(550) is retardation in plane at 550 nm, of 1.1 to 7.0.

11. The optically anisotropic film of claim 5, having an Nz value, where Nz=Rth(550)/Re(550)+0.5, Rth(550) is retardation along thickness direction at 550 nm, and Re(550) is retardation in plane at 550 nm, of 0.1 to 0.9.

12. A liquid crystal cell substrate, comprising a substrate and an optically anisotropic film as set forth in claim 1.

13. A liquid crystal display device comprising an optically anisotropic film as set forth in claim 1.

14. The liquid crystal display device of claim 13, being a VA-mode liquid crystal display device.

15. The liquid crystal display device of claim 13, being an IPS-mode liquid crystal display device.

16. The liquid crystal display device of claim 13, wherein the optically anisotropic film is disposed in a liquid crystal cell.

17. The liquid crystal display device of claim 13, where the optically anisotropic film is disposed in a liquid crystal cell, as being formed in the regions corresponded to the individual pixels.

18. A liquid crystal display device comprising an optically anisotropic film as set forth in claim 7 as a first optically anisotropic layer, and a second optically anisotropic layer having Rth (550) of 20 to 300 nm.

19. A method of producing an optically anisotropic film comprising irradiating a composition with polarized light, so that birefringence develops in the composition,
wherein the composition comprises at least one compound having a partial structure represented by formula (1) below: 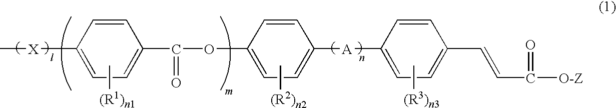
where, each of R¹, R² and R³ independently represent a substituent; X represents a divalent linking group selected from Linking Group I shown below, or a divalent linking group formed by combining two or more species selected from Linking Group I shown below; “A” represents —COO—, —OCO—, or a substituted or non-substituted phenylene group, oxadiazole group or alkynylene group; Z represents a substituted or non-substituted alkyl group or aryl group; each of n1, n2 and n3 represents an integer of 0 to 4; and each of l, m and n represents an integer of 0 to 4;
Linking Group I:
single bond, —O—, —CO—, —NR⁶— (R⁶ represents a hydrogen atom, alkyl group or aryl group), —S—, —SO₂—, —P(═O)(OR⁷)— (R⁷ represents an alkyl group or aryl group), alkylene group and arylene group.

20. A method of producing an optically anisotropic film comprising disposing a composition on a rubbed surface; and irradiating the composition with polarized light in a direction different from the rubbing direction of said rubbed surface, so that birefringence develops in the composition
wherein the composition comprises at least one compound having a partial structure represented by formula (1) below: 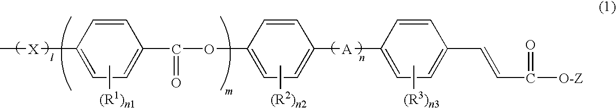
where, each of R¹, R² and R³ independently represent a substituent; X represents a divalent linking group selected from Linking Group I shown below, or a divalent linking group formed by combining two or more species selected from Linking Group I shown below; “A” represents —COO—, —OCO—, or a substituted or non-substituted phenylene group, oxadiazole group or alkynylene group; Z represents a substituted or non-substituted alkyl group or aryl group; each of n1, n2 and n3 represents an integer of 0 to 4; and each of l, m and n represents an integer of 0 to 4;
Linking Group I:
single bond, —O—, —CO—, —NR⁶— (R⁶ represents a hydrogen atom, alkyl group or aryl group), —S—, —SO₂—, —P(═O)(OR⁷)— (R⁷ represents an alkyl group or aryl group), alkylene group and arylene group.

21. A polymer comprising at least one repeating unit represented by formula (2): 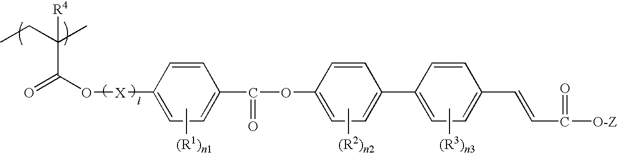
where, each of R¹, R² and R³ independently represent a substituent; R⁴ represents a hydrogen atom or substituent; X represents a divalent linking group selected from Linking Group I shown below, or a divalent linking group formed by combining two or more species selected from Linking Group I shown below; Z represents a substituted or non-substituted alkyl group or aryl group; each of n1, n2 and n3 represents an integer of 0 to 4; and each of l, m and n represents an integer of 0 to 4;
Linking Group I:
single bond, —O—, —CO—, —NR⁶— (R⁶ represents a hydrogen atom, alkyl group or aryl group), —S—, —SO₂—, —P(═O)(OR⁷)— (R⁷ represents an alkyl group or aryl group), alkylene group and arylene group.

22. The polymer of claim 21, further comprising a repeating unit represented by formula (5) and/or formula (7) below: 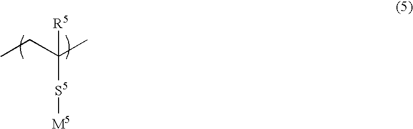
where, R⁵ represents a hydrogen atom or substituent, S⁵ represents a divalent linking group, and M⁵ represents a mesogen group; 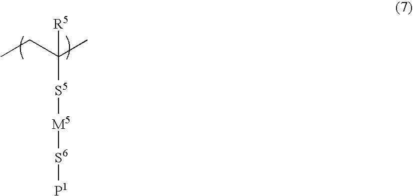
where, R⁵ represents a hydrogen atom or substituent, S⁵ represents a divalent linking group, M⁵ represents a mesogen group, S⁶ represents a divalent linking group, and P¹ represents a polymerizable group.
